(19) United States
(12) Patent Application Publication
(10) Pub. No.: US 2015/0010131 A1
(43) Pub. Date: Jan. 8, 2015

(54) X-RAY APPARATUS AND X-RAY IMAGE DIAGNOSTIC APPARATUS

(71) Applicants: Kimitaka Arisaka, Tokyo (JP); Atsushi Hibino, Tokyo (JP); Tetsuji Sairaiji, Tokyo (JP)

(72) Inventors: Kimitaka Arisaka, Tokyo (JP); Atsushi Hibino, Tokyo (JP); Tetsuji Sairaiji, Tokyo (JP)

(21) Appl. No.: 14/378,399

(22) PCT Filed: Feb. 27, 2013
(86) PCT No.: PCT/JP2013/055075

§ 371 Date: Aug. 13, 2014

(30) Foreign Application Priority Data
Feb. 29, 2012 (JP) 2012-043902

(51) Int. Cl. A61B 6/00

(52) U.S. Cl. CPC A61B 6/4488; A61B 6/4441; A61B 6/4405; A61B 6/5211
USPC 378/62; 378/199; 378/197

(57) ABSTRACT

In order to provide an X-ray apparatus and an X-ray image diagnostic apparatus that reduce uncomfortable feeling due to exhaust heat generated when air-cooling an X-ray tube device, the X-ray image diagnostic apparatus is comprised of an X-ray tube, the X-ray tube device 102 including the X-ray tube device housing that accommodates the X-ray tube, the X-ray detector 103 that detects an X-ray generated from the X-ray tube device, and the arm 101 that supports the X-ray tube device 102 and the X-ray detector 103 with them facing each other, the X-ray tube device 102 and the X-ray detector 103 are located respectively on an end side and the other end side of the arm 101, and the first air flow path 104 where air passed through the outer surface of the X-ray tube device housing 402 flows inside the arm 101 is formed.

### TECHNICAL FIELD

The present invention relates to an X-ray apparatus and X-ray image diagnostic apparatus, and in particular, to a technique for cooling an X-ray tube device.

### BACKGROUND ART

In recent years, as the IVR procedure (Interventional Radiology: a procedure to perform medical treatment using an X-ray fluoroscopic image) increases, a mobile X-ray fluoroscopic imaging apparatus that can be used with a large power output and for a longer time and that includes a small and highly reliable X-ray tube device is needed. For such an X-ray tube device, how effectively heat generated from the X-ray tube is released to the outside of the housing is important to enhance the reliability.

In PTL 1, a mobile X-ray fluoroscopic imaging apparatus that includes a coolant reservoir tank detachably disposed inside the main body supporting the C-arm and a coolant circulation passage along the C-arm as well as that cools an X-ray irradiation unit by circulating coolant between the X-ray irradiation unit and the coolant reservoir tank is disclosed.

### CITATION LIST

### Patent Literature

PTL 1: Japanese Unexamined Patent Application Publication No. 2011-062240

### SUMMARY OF INVENTION

### Technical Problem

However, because a mobile X-ray fluoroscopic imaging apparatus described in PTL 1 must install a coolant reservoir tank inside the main body that supports the C-arm, a space for the installation is required, resulting in a problem being an obstacle for downsizing. Also, in case of installing a heat exchanger separately, the additional space is required. Moreover, there is a problem of a risk of coolant leakage caused by breakage.

On the other hand, if an X-ray tube device is cooled using an air cooling mechanism to solve the above problem, there is a problem where exhaust with a temperature higher than a room temperature is emitted.

The present invention was made to improve such problems and is aimed to provide a technique for reducing high-temperature exhaust emission due to heat generated when air-cooling an X-ray tube device.

### Solution to Problem

In order to solve the above problem, in the present invention, air warmed by heat generated from an X-ray tube is cooled by passing through the inside of an arm that supports the X-ray tube.

### Advantageous Effects of Invention

According to the present invention, a technique for reducing high-temperature exhaust emission can be provided by heat generated when air-cooling an X-ray tube device.

### BRIEF DESCRIPTION OF DRAWINGS

FIG. 1 is an explanatory diagram showing an overview of a mobile X-ray fluoroscopic imaging apparatus related to the present embodiment.

FIG. 2 is an explanatory diagram showing an outline shape of an X-ray tube device housing, (a) shows a state where an X-ray tube is accommodated in the X-ray tube device housing, and (b) shows a state where a housing cover is placed on the housing.

FIG. 3 is an explanatory diagram showing an overview of a heat-release mechanism.

FIG. 4 is an explanatory diagram where the vicinity of an X-ray detector in FIG. 3 is magnified.

FIG. 5 is an explanatory diagram showing an overall configuration of the C-arm 101.

FIG. 6 is a perspective diagram where the end of an X-ray tube device side in the C-arm 101 is magnified.

FIG. 7 is a cross-sectional diagram of the C-arm 101 on the surface A in FIG. 5.

FIG. 8 is a perspective diagram where the end of an X-ray detector side in the C-arm 101 is magnified.

FIG. 9 is a cross-sectional diagram of the C-arm 101 on the surface B in FIG. 5.

FIG. 10 is a perspective diagram showing an overall configuration of the vicinity of an X-ray tube device in the mobile X-ray device 2.

FIG. 11 is a side-view diagram showing an overall configuration of an X-ray tube device housing and an air guide cover in the mobile X-ray device 2.

FIG. 12 is a top-view diagram showing an overall configuration in the vicinity of an X-ray tube device in the mobile X-ray device 2.

FIG. 13 is a front-view diagram showing an overall configuration in the vicinity of an X-ray tube device in the mobile X-ray device 2.

FIG. 14 is an explanatory diagram showing an air flow path in a case where there are no bent portions of the housing cover.

FIG. 15 is a perspective diagram showing a structure in the vicinity of the X-ray tube device 102 of the C-arm 101 before an air guide cover is installed.

FIG. 16 a perspective diagram showing a structure in the vicinity of the X-ray tube device 102 of the C-arm 101 after an air guide cover is installed and shows a state where the air guide cover 302 is viewed from the C-arm 101 side.

FIG. 17 is a perspective diagram showing a structure in the vicinity of the X-ray tube device 102 of the C-arm 101 after an air guide cover is installed and shows a state where the air guide cover 302 is viewed from the X-ray tube device 102 side.

FIG. 18 is an explanatory diagram showing an overview of a heat-release mechanism of a mobile X-ray fluoroscopic imaging apparatus related to the second embodiment.

FIG. 19 is an explanatory diagram where the vicinity of an exhaust outlet in the C-arm 101 is magnified.

FIG. 20 is an explanatory diagram showing a transformed state after the C-arm 101 is Slid.

FIG. 21 is an explanatory diagram showing an overview of a heat-release mechanism of a mobile X-ray fluoroscopic imaging apparatus related to the fifth embodiment.

FIG. 22 is a perspective diagram where the vicinity of an X-ray detector side in the C-arm 101 is magnified.

FIG. 23 is a front-view diagram where the vicinity of an X-ray detector side in the C-arm 101 is magnified.

FIG. 24 is an explanatory diagram where the vicinity of an X-ray tube device side in the C-arm 101 is magnified.

### DESCRIPTION OF EMBODIMENTS

The present embodiment includes an X-ray tube device with an X-ray tube device housing that accommodates an X-ray tube and the X-ray tube, an X-ray detector that detects an X-ray generated from the X-ray tube device, and an arm that supports the X-ray tube device on one end side and the X-ray detector on the other end side facing with each other and relates to an X-ray apparatus that is characterized by that a first air flow path where air passed through an outer surface of the X-ray tube device housing flows is formed inside the arm.

Also, a heat-release fin having a fin group in which the other end becomes an open end with one end contacting the outer surface is mounted on an outer surface of the X-ray tube device housing, and a second air flow path that is a flow path where air passes through the fin group and that continuously connects to the first air flow path may be formed in the X-ray tube device.

Also, the X-ray apparatus further has a housing cover that covers a surface facing toward the X-ray detector in the X-ray tube device housing, the housing cover has an open end contact section that contacts an open end of the fin group on a surface facing toward the X-ray tube device, and the second air flow path may be formed between a surface of the X-ray tube device housing on which the heat-release fin is mounted and a surface that includes the open end contact section in the housing cover.

Also, the X-ray apparatus may further have an air guide cover that covers an end of the first air flow path side in the second air flow path to an end of the X-ray tube device side in the first air flow path.

Also, the X-ray apparatus is installed between the second air flow path and the first air flow path and further includes a fan that specifies a blowing direction in the first air flow path as well as a fan supporting section that supports the fan, the fan supporting section is mounted on the end of the first air flow path side in the air guide cover, and then an insulating and sealing member may be placed to connect between the fan supporting section and an end of the first air flow path.

Also, an X-ray tube device cover that covers the X-ray tube device is mounted on an end of the arm, the X-ray tube device cover has a cover opening that continuously connects an internal space of the X-ray tube device cover to an external space of the X-ray tube device cover, the second air flow path continuously connects to an internal space of the X-ray tube device cover on an end opposite to the first air flow path side in the second air flow path, and the first air flow path may be continuously connected to an external space of the X-ray tube device cover via the second air flow path, an internal space of the X-ray tube device cover, and the cover opening.

Also, an X-ray detector cover that covers the X-ray detector is mounted with a gap where air flows between the arm on the other end of the arm, the first air flow path has an opening that continuously connects to an internal space of the X-ray detector cover on an end of the X-ray detector side in the first air flow path, and the first air flow path may be continuously connected to an external space of the X-ray detector cover via the opening, an internal space of the X-ray detector cover, and the gap.

Also, in order to reverse the positions in the vertical direction of the X-ray tube device and the X-ray detector, an arm supporting unit that supports the arm rotatably is further mounted, and the X-ray apparatus may further have a fan that specifies a blowing direction in the first air flow path, a posture detection unit that detects a posture of the arm, and a control unit that controls a blowing direction of the fan so that air blows from a position side relatively close to a floor toward a side close to a ceiling inside the first air flow path based on a detection result by the posture detection unit.

Also, the fan is a forward-backward rotatable fan that can rotate both forward and backward, and the control unit performs switching control for forward rotation or backward rotation of the fan based on the detection result.

Also, the fan includes a first fan that blows air in the first air flow path from the X-ray tube device side to the X-ray detector side and a second fan that blows air from the X-ray detector side to the X-ray tube device side, and the control unit may select either the first fan or the second fan to rotate it based on the detection result.

Also, a fan rotation supporting section that supports the fan rotatably in order to reverse the blowing direction is further provided for fixtures that fix the fan, and the control unit may perform rotation control for the fan rotation supporting section based on the detection result.

The arm is an arc-shaped C-arm that supports the X-ray tube device and the X-ray detector with them facing each other and has an arm supporting unit that supports the C-arm slidably along the arc as well as an arm opening that continuously connects the first air flow path to an external space of the C-arm in the vicinity of the arm supporting unit in the C-arm, and bellows sections that are stretched with the slide movement may be mounted on the X-ray tube device side and the X-ray detector side respectively across the arm opening.

Also, the X-ray apparatus further has an X-ray tube device cover that covers the X-ray tube device and a fan that controls a blowing direction in the first air flow path, the first air flow path and a third air flow path that is continuously connected to an internal space of the X-ray tube device cover isolated from the first air flow path are provided inside the arm, the first air flow path and the third air flow path are sealed so that they are not continuously connected to the outside of the arm, an insulation process is performed for either surface of the first air flow path or the third air flow path, and a continuous connecting section where the first air flow path and the third air flow path are continuously connected may be provided on the end of the X-ray detector side inside the arm.

Also, the heat-release fin is molded and integrated with the X-ray tube device housing, or the heat-release fin may be comprised separately from the X-ray tube device housing and be fixed so that the heat-release fin contacts the outer surface of the X-ray tube device housing.

Also, the X-ray tube device may be fixed in a state where a heat-release sheet with insulation properties is tucked on an end side of the arm.

Also, the X-ray apparatus may be comprised of a main body equipped with the arm and a moving section that moves the main body on a floor.

Additionally, an X-ray image diagnostic apparatus related to the present embodiment is comprised of any one of the above X-ray apparatuses, an image processing unit that generates an X-ray image based on an X-ray detected by the X-ray detector, and an image display unit that displays the X-ray image.

The image processing unit may generate an X-ray image being comprised of dynamic and static images of the object.

Also, an X-ray apparatus related to the present embodiment is comprised of an X-ray tube, an X-ray tube device equipped with an X-ray tube device housing that accommodates the X-ray tube, and an arm that supports the X-ray tube device and forms an air flow path in which air that passed through the outer surface of the X-ray tube device housing flows.

Hereinafter, referring to figures, the above embodiment will be described in detail. Additionally, through all the figures, the same numerals are provided for components having the same functions, and the repeated explanations thereof will be omitted. Hereinafter, as an example of an X-ray image diagnostic apparatus, a mobile X-ray fluoroscopic imaging apparatus that generates an X-ray image of dynamic images by fluoroscoping an object and an X-ray image of static images by imaging an object will be described.

### First Embodiment

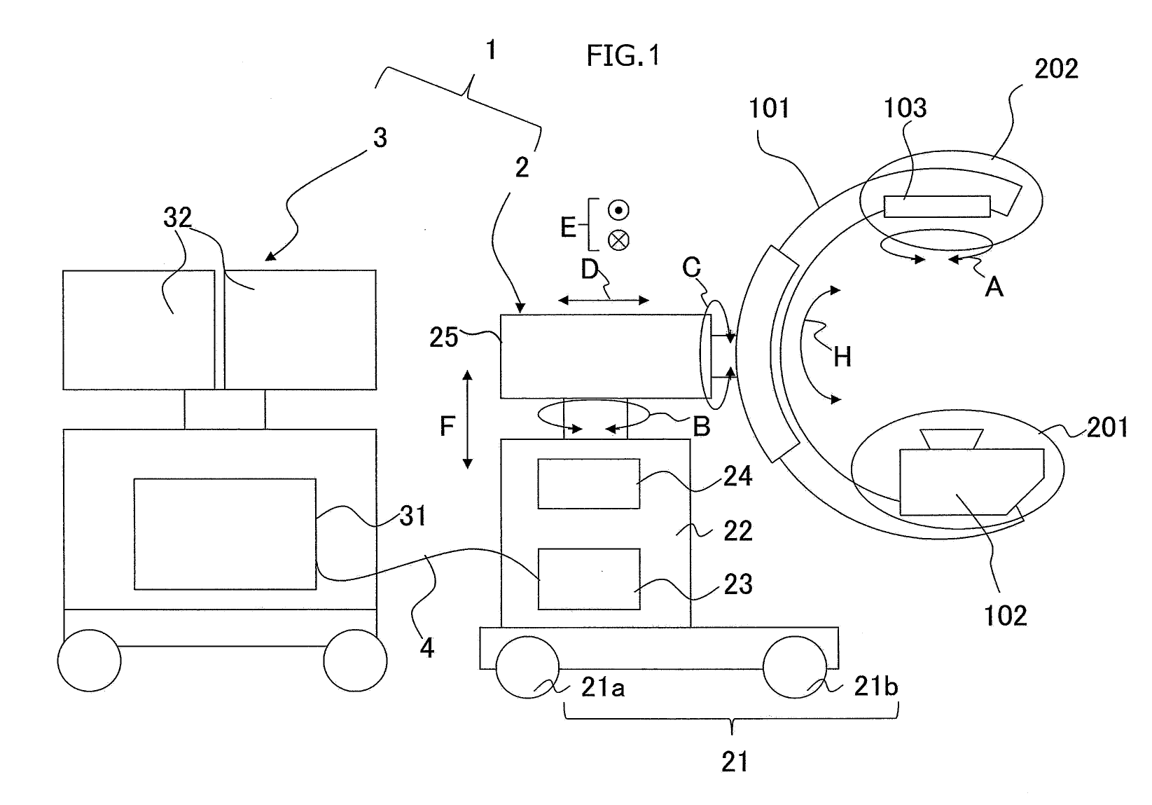

First, based on FIG. 1, the overall configuration of a mobile X-ray fluoroscopic imaging apparatus related to the present embodiment will be described. FIG. 1 is an explanatory diagram that shows an overview of a mobile X-ray fluoroscopic imaging apparatus related to the present embodiment.

As shown in FIG. 1, the mobile X-ray fluoroscopic imaging apparatus 1 is largely comprised of the mobile X-ray device 2 including the X-ray tube device 102 as well as the X-ray detector 103 and the mobile image display device 3 including the display 32 that displays an X-ray image. The mobile X-ray device 2 and the mobile image display device 3 are configured separately and electrically connected with the cable 4. Although, in the mobile X-ray fluoroscopic imaging apparatus 1 related to the present embodiment, the mobile X-ray device 2 and the mobile image display device 3 are configured separately, the mobile X-ray fluoroscopic imaging apparatus may be the one in which an image display device is integrated with the mobile X-ray device 2.

The mobile X-ray device 2 is comprised of the arm 101 that connects and supports the X-ray tube device 102 and the X-ray detector 103 with them disposed oppositely, the main body 22 mounted with the arm supporting unit 25 that connects the arm 101 to the main body 22 rotatably and movably, and the traveling unit 21 that moves the main body on a floor. The traveling unit 21 has the two wheels 21a and 21b.

In the mobile X-ray device 2 related to the present embodiment, although the arm 101 has an arc-shape i.e., an approximate C-shape and is especially referred to as the C-arm 101 in the following explanations, the arm is not limited to a C-shape as long as the arm is the one that connects and supports the X-ray tube device 102 and the X-ray detector 103 with them disposed oppositely.

On an end of the C-arm 101, the X-ray tube device 102 is mounted and fixed on the C-arm 101. The details of the fixing structure will be described later. The X-ray tube device 102 and an end of the C-arm 101 are covered with the X-ray tube device cover 201. In a position opposite to the X-ray detector 103 in the X-ray tube device 102, an X-ray diaphragm that limits an irradiation region of an X-ray irradiated from an X-ray tube is installed.

The X-ray detector 103 detects an X-ray generated from the X-ray tube device 102 and outputs an electrical signal according to the X-ray intensity. Although the X-ray detector 103 is composed of a flat panel detector (hereinafter, abbreviated as “FPD”) in the present embodiment, the device may be the one in which an image intensifier is used and is not limited to FPD as long as a function to detect an X-ray is included. The X-ray detector 103 is connected to the other end of the C-arm 101 rotatably on a floor (shown in the arrow A direction in the figure). The X-ray detector cover 202 is mounted on the other end of the C-arm 101, and the X-ray detector 103 is disposed inside the cover.

The arm supporting unit 25 supports the C-arm 101 rotatably and movably for the main body 22. Rotation directions include those on a horizontal surface (shown in the arrow B direction in FIG. 1) and on a vertical surface (shown in the arrow C direction in FIG. 1). When rotating along in the arrow C direction, positions along in the vertical direction of the X-ray tube device 102 and the X-ray detector 103 are reversed.

Also, movement directions include a back-and-forth direction based on the main body 22 on a horizontal surface (shown in the arrow D direction in the figure) and a direction orthogonal to the back-and-forth direction based on the main body 22 (shown in the arrow E direction in the figure). Additionally, the movement directions include an up-and-down direction based on the main body 22 (shown in the arrow F direction in the figure).

Also, the arm supporting unit 25 slides the C-arm 101 along the arc of the C-arm 101 (shown in the arrow H direction in the figure).

The main body is comprised of the X-ray tube device 102, the X-ray detector 103, the control unit 23 that controls operations of the respective components including the arm supporting unit 25, and the operation unit 24 that accepts input operations by an operator. The control unit 23 and the operation unit 24 are electrically connected, and the control unit 23 performs operation control of X-ray irradiation, controls detection of an X-ray transmitted through an object (hereinafter, abbreviated as “transmitted X-ray”) and reading electrical signals, and controls rotation and movement operations of the C-arm 101 based on an input signal input from the operation unit 24. Additionally, the C-arm 101 may rotate and move according to control by the control unit 23 and may rotate and move by operator's manual operation.

The traveling unit 21 may have a driving device in which a motor drives the wheels 21a and 21b as well as a braking device in addition to the two wheels 21a and 21b. Although a motor may drive both the wheels 21a and 21b, the motor may drive only one wheel, and the other wheel may be comprised of wheeled casters.

The mobile image display device 3 obtains an electrical signal corresponding to a transmitted X-ray read from the X-ray detector 103 according to operation control of the control unit 23 via the cable 4, is comprised of the image processing unit 31 that generates an X-ray image of an object based on the electrical signal, CRT, a liquid crystal panel, etc., and has the display 32 that displays an X-ray image of an object. The display 32 of the mobile image display device 3 related to the present embodiment may have not only two displays, but also only one display. Also, a design change such as where the image processing unit 31 is mounted on the mobile X-ray device 2 to configure the mobile image display device 3 so that a generated X-ray image is output is available, and the control unit 23, the operation unit 24, and the image processing unit 31 may be mounted on either the mobile X-ray device 2 or the mobile image display device 3.

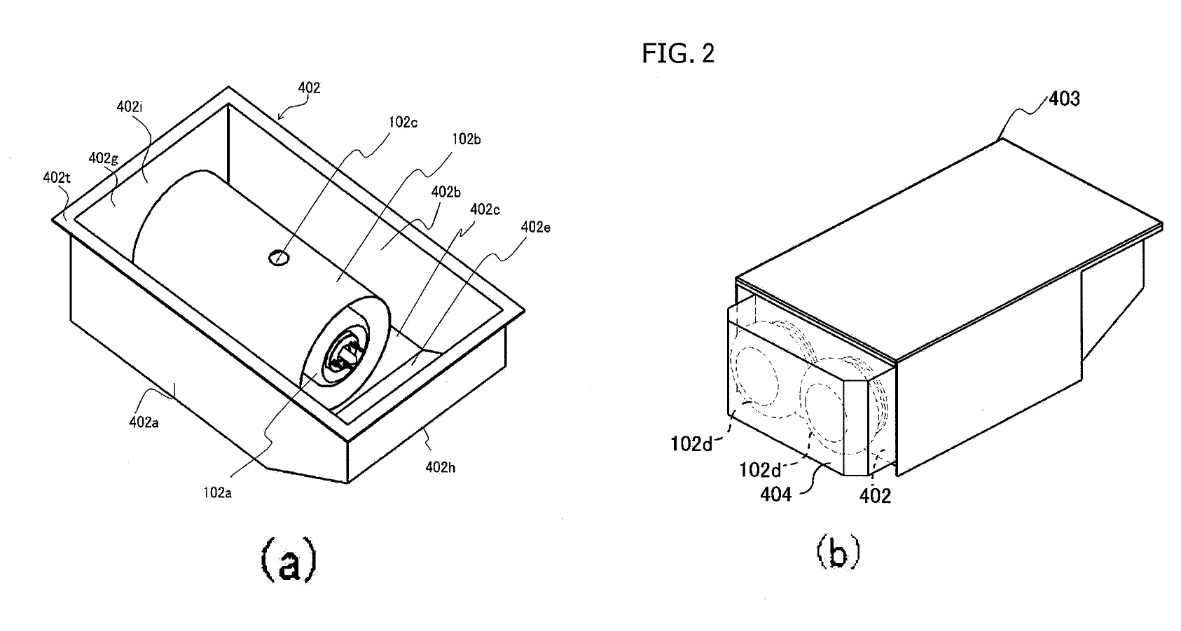

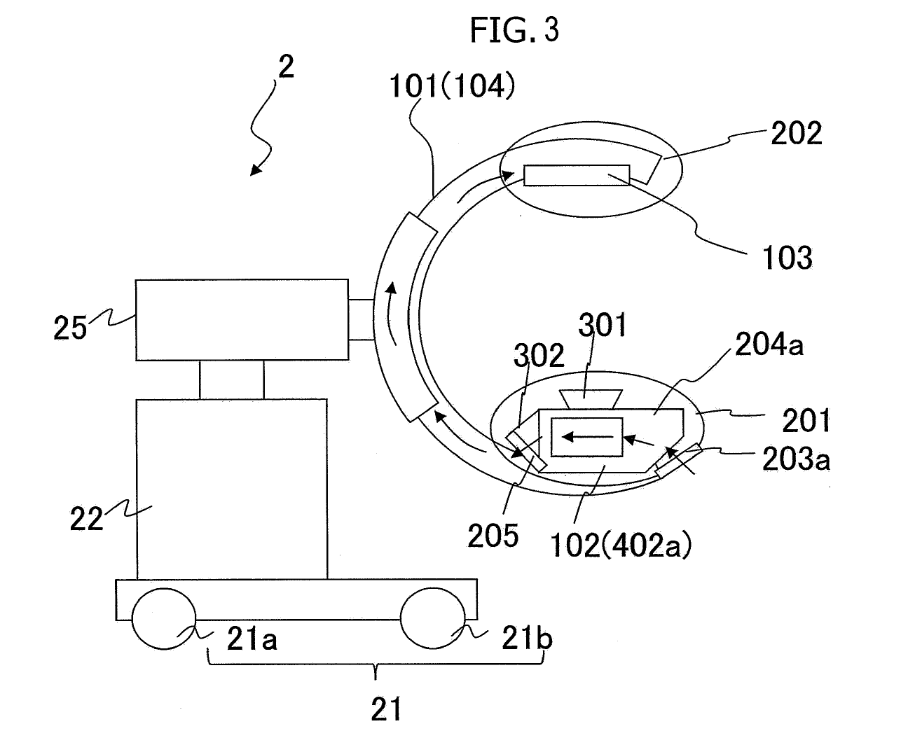

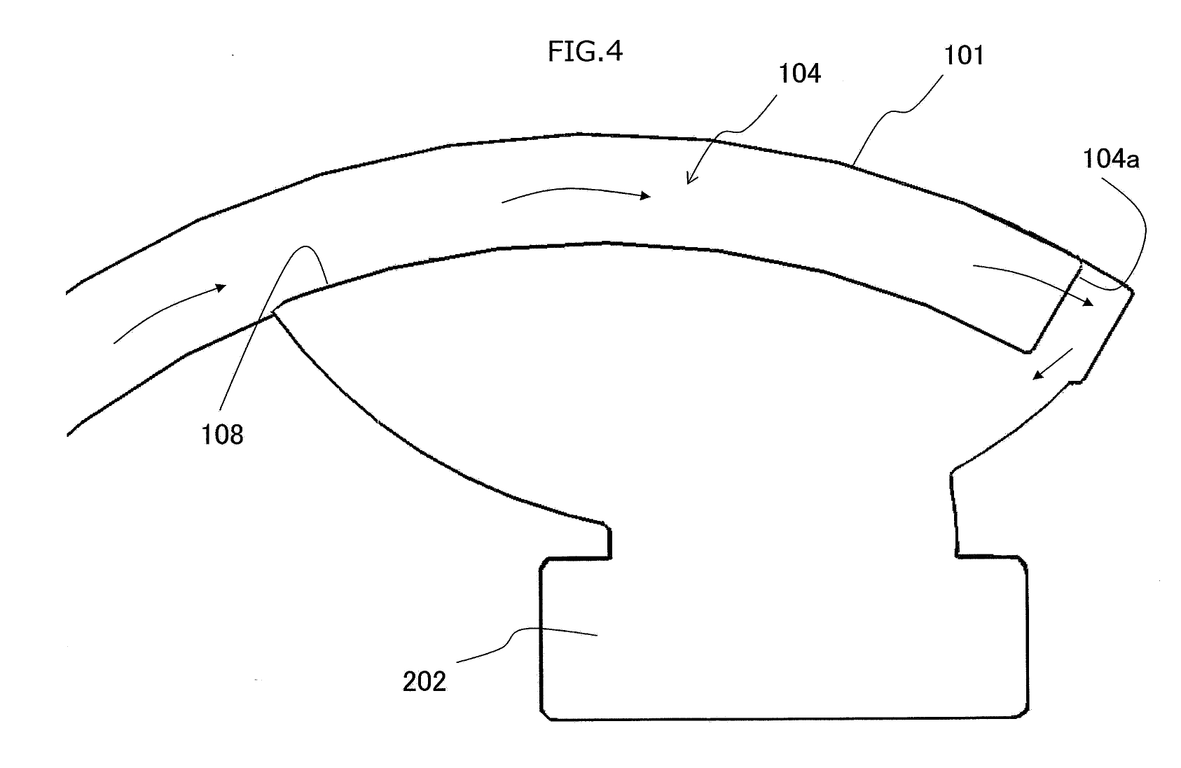

The mobile X-ray device 2 has a heat-release structure to release heat generated from the X-ray tube device 102. An overview of the heat-release structure is shown in FIGS. 2 to 4. FIG. 2 is an explanatory diagram showing an outline shape of an X-ray tube device housing, (a) shows a state where an X-ray tube is accommodated inside the X-ray tube device housing, and (b) shows a state where the housing cover is placed. FIG. 3 is an explanatory diagram showing an overview of the heat-release structure. FIG. 4 is an explanatory diagram where the vicinity of an X-ray detector in FIG. 3 is enlarged.

The X-ray tube device 102 includes a box-shaped X-ray tube device housing with an opening surface and a housing cover that covers the opening surface.

As shown in (a) of FIG. 2, the X-ray tube device housing 402 is comprised of the rectangular surface (hereinafter, referred to as “bottom surface”) 402c that contacts the C-arm 101 when placed on the C-arm 101 and located on the opposite side to the X-ray detector 103 side in the X-ray tube device 102, the rectangular inclined surface 402e inclines toward the X-ray detector 103 side from a short side of the bottom surface 402c, the first side surface 402a that stands (also referred to as “protrudes”) toward the X-ray detector 103 side from a long side of the bottom surface 402c and a side of the inclined surface 402e immediately following the long side, and the second side surface 402b that stands facing the first side surface 402a and toward the X-ray detector 103 side from the other long side of the bottom surface 402c and a side of the inclined surface 402e immediately following the long side.

Additionally, the X-ray tube device housing 402 includes the third side surface 402h that stands toward the X-ray detector 103 side from a short side opposite to the bottom surface 402c of the inclined surface 402e and the forth side surface 402i that stands facing the third side surface 402h and toward the X-ray detector 103 side from the other short side of the bottom surface 402c. The X-ray tube device housing 402 further includes the facing surface 402t facing the X-ray tube device 102. The facing surface 402t has the opening 402g to dispose the X-ray tube 102a inside the X-ray tube device housing 402.

Inside the X-ray tube device housing 402, the X-ray tube 102a including a vacuum container that accommodates the anode and cathode in a vacuum state and the passive device (not shown in the figure) are set. The periphery of the X-ray tube 102a is comprised of an X-ray shield material (such as lead) and is covered with the tube pipe 102b that shields unnecessary X-rays. The tube pipe 102b has the X-ray radiation aperture 102c comprised of an opening in order to release X-rays generated from the X-ray tube 102a.

As shown in (b) of FIG. 2, the X-ray tube 102a and the passive device are accommodated inside the X-ray tube device housing 402 in a state filled with insulating oil and are sealed with the housing cover 403. Additionally, the X-ray tube device housing 402 related to the present embodiment includes the outer member 102d of the X-ray tube 102a outside the forth side surface 402i. The outer member 102d is surrounded with the protection cover 404 that protects the outer member 102d. The protection cover 404 protrudes from the housing cover 403 in a state where the housing cover 403 is placed on the X-ray device housing 420. The outer surface (the opposite surface to a surface facing the outer member 102d) of the protection cover 404 is a wall surface of an air guide path to be described later.

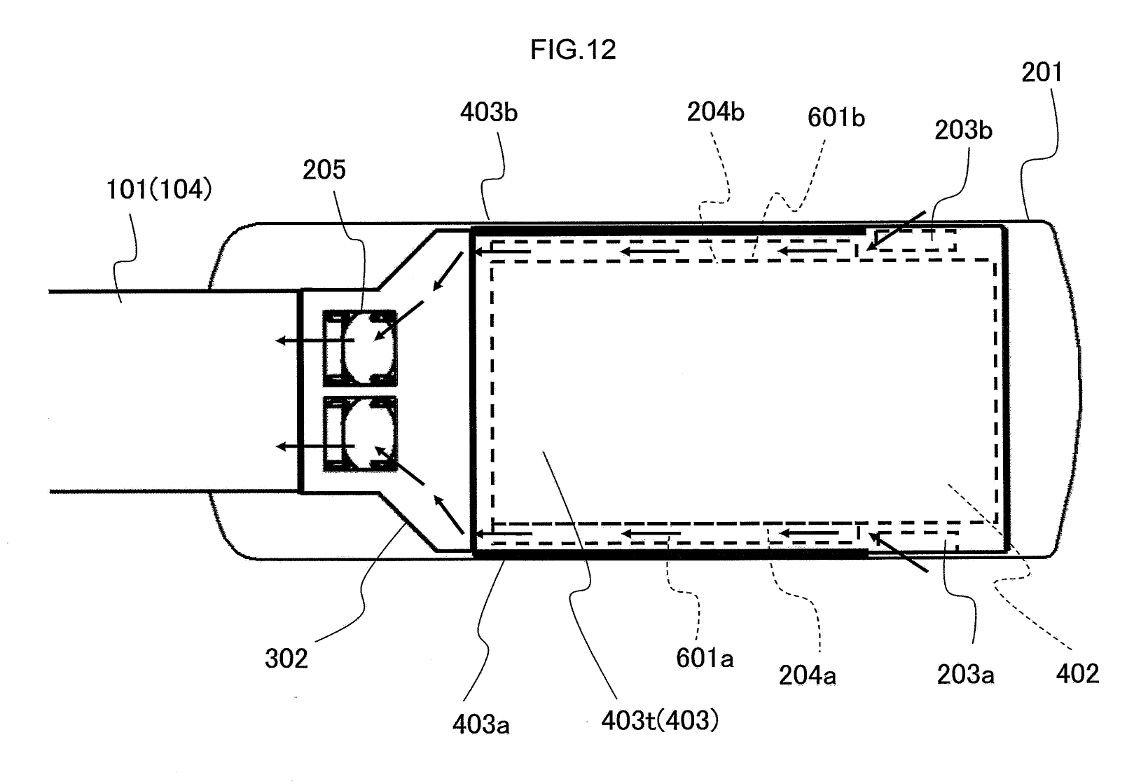

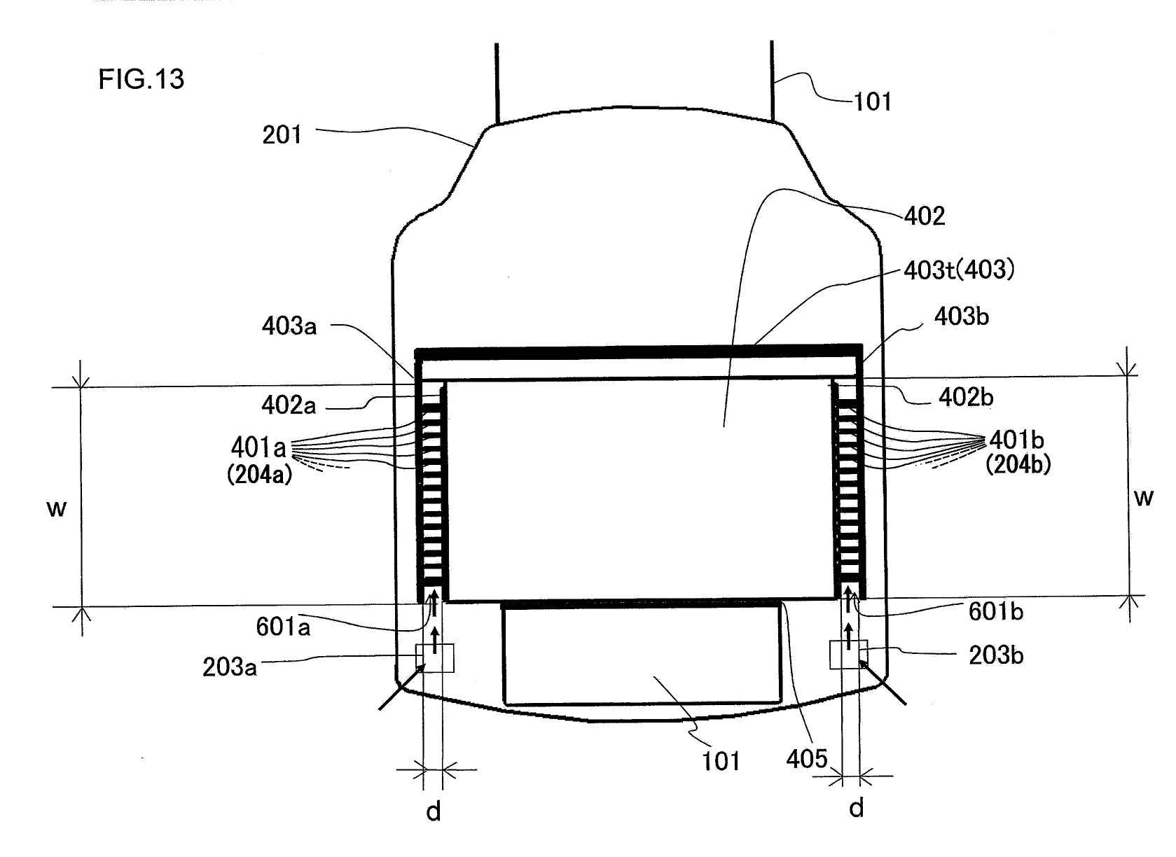

A heat-release structure, as shown in FIG. 3, is largely comprised of the cover opening 203a created on the X-ray tube device cover 201, the heat-release fin 204a created on the first side surface 402a of the X-ray tube device housing 402 in the X-ray tube device 102, the fan 205, and the first air flow path 104 where air blown from the fan 205 flows inside the C-arm 101. Although not shown in FIG. 3, the heat-release fin 204b is mounted also on the second side surface 402b of the X-ray tube device housing 402 similarly to the first side surface 402a (see FIG. 13). Among the heat-release fins 204a and 204b and the fan 205, the air guide cover 302 to guide air passed through the heat-release fins 204a and 204b to the fan 205 is mounted. Via the cover opening 203a, the internal space and external space of the X-ray tube device cover 201 are continuously connected. Although not shown in FIG. 3, the cover opening 203b similar to the cover opening 203a is mounted in a position close to the second side surface 402b (see FIG. 12).

Also, as shown in FIG. 4, the X-ray detector cover 202 is mounted on the other end of the C-arm 101 in a state where the gap 108 in which air flows is created. The first air flow path 104 has the opening 104a that continuously connects to the internal space of the X-ray detector cover 202. Via the gap 108, the internal space and external space of the X-ray tube device cover 201 are continuously connected.

As described above, in the heat-release structure, air basically flows into the inside of the X-ray tube device cover 201 from the cover openings 203a and 203b, is heated by passing through the heat-release fins 204a and 204b, passes through an air guide path created between the air guide cover 302 and the protection cover 404, and then flows into the fan 205. Then, the heated air is emitted inside the first air flow path 104 by the fan 205 and is cooled by a housing of the C-arm 101 while passing through the first air flow path 104.

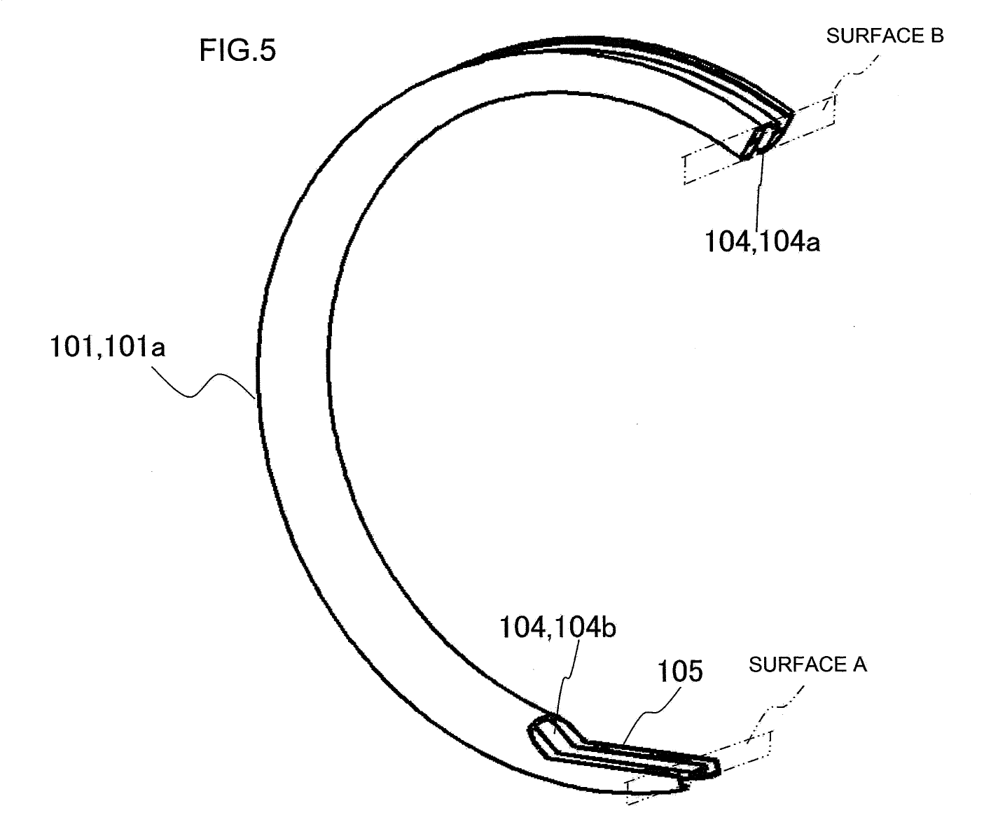

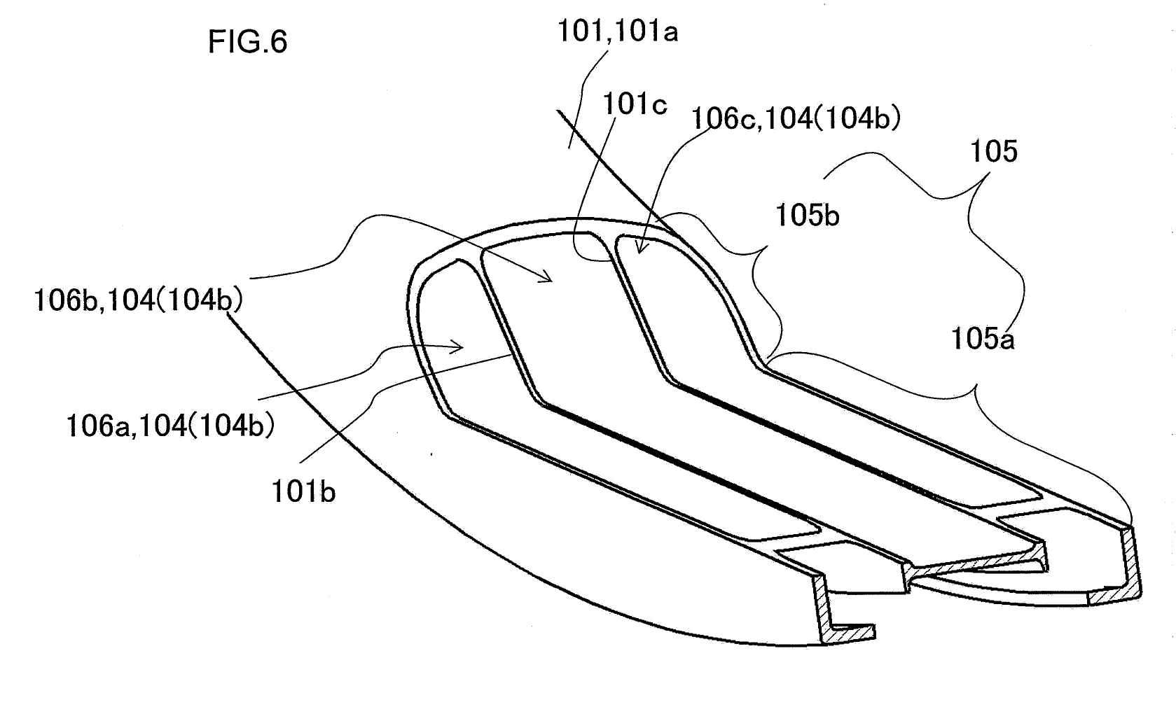

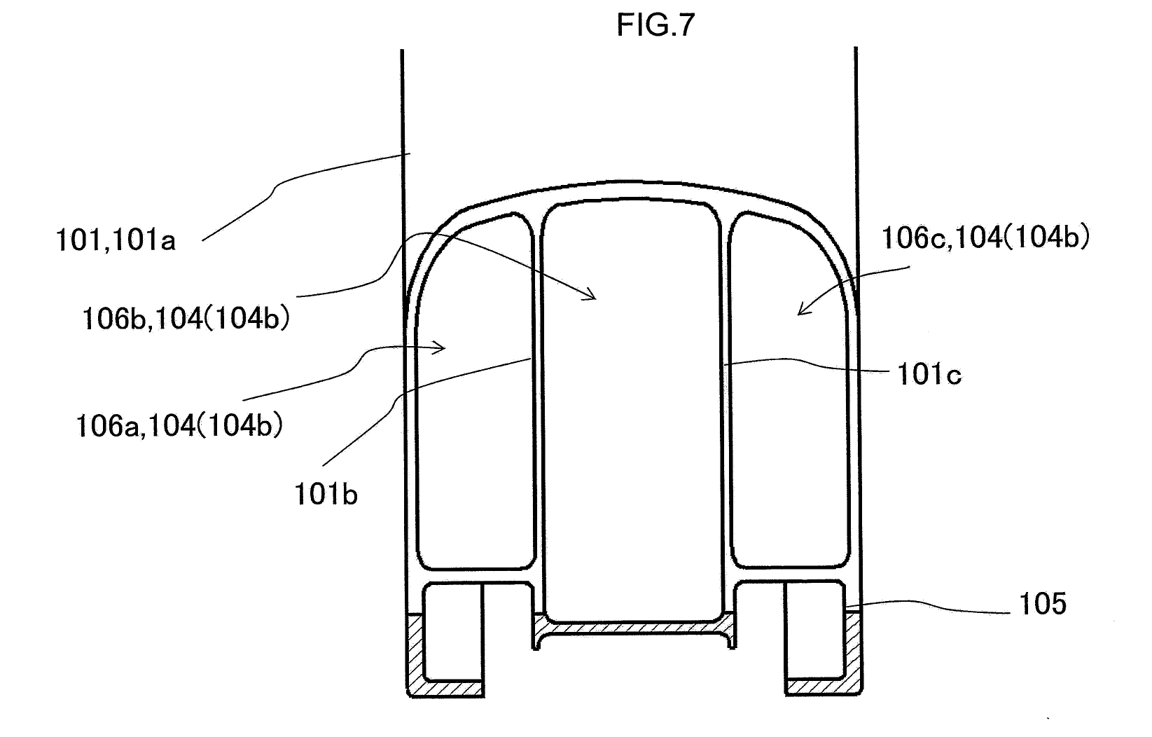

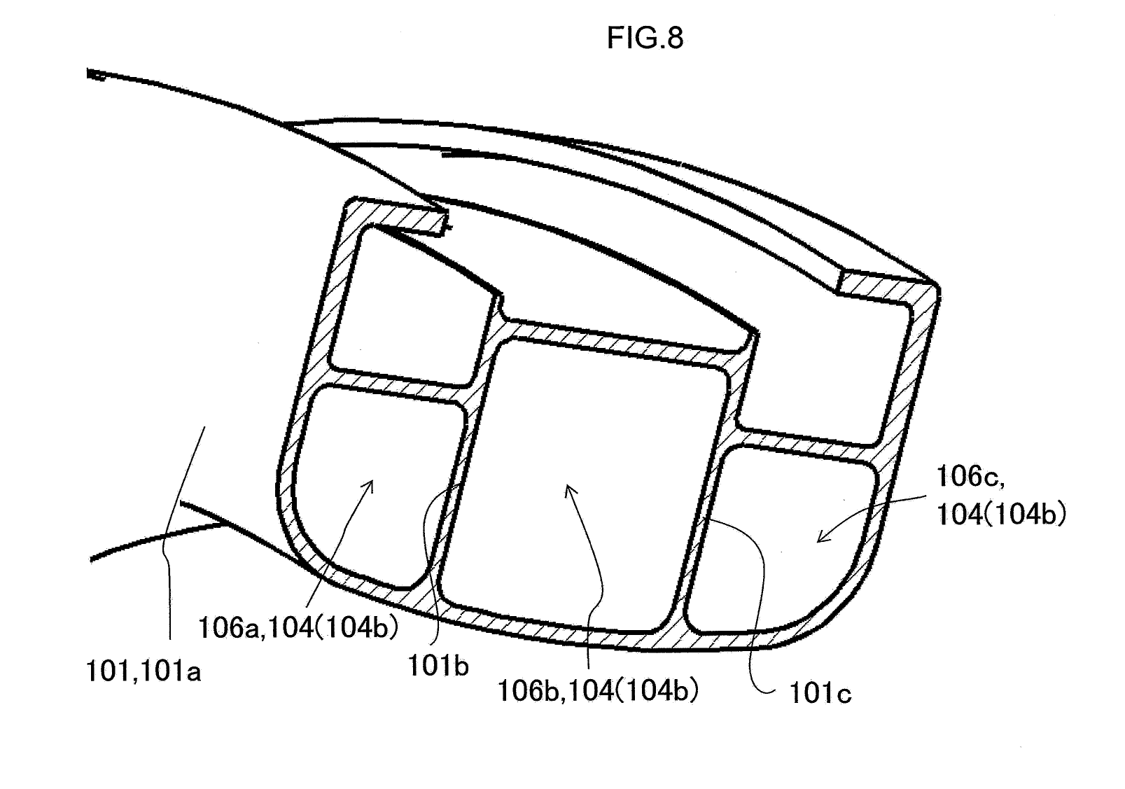

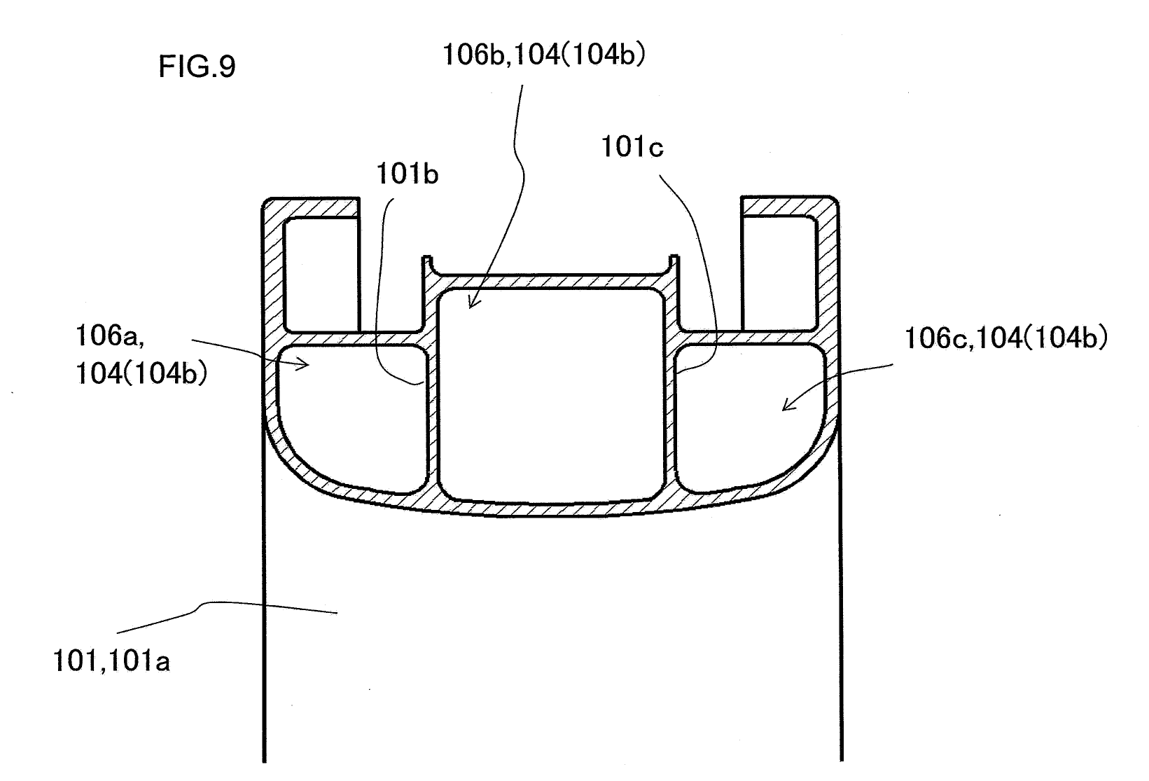

Next, the configuration of the C-arm will be described based on FIGS. 5 to 9. FIG. 5 is an explanatory diagram showing an overall configuration of the C-arm 101. FIG. 6 is a perspective diagram where the end of an X-ray tube device side in the C-arm 101 is magnified. FIG. 7 is a cross-sectional diagram of the C-arm 101 on the surface A in FIG. 5. FIG. 8 is a perspective diagram where the end of an X-ray detector side in the C-arm 101 is magnified. FIG. 9 is a cross-sectional diagram of the C-arm 101 on the surface B in FIG. 5. Additionally, in FIGS. 6 and 7, a cross-sectional position on the surface A in FIG. 5 is shown using oblique lines. In FIGS. 8 and 9, a cross-sectional position on the surface B in FIG. 5 is shown using oblique lines.

As shown in FIG. 5, the C-arm 101 is formed into an approximate arc including the first air flow path 104 comprised of a hollow space in the inside surrounded with the outer surface 101a. The cutout 105 to place and fix the X-ray tube device housing 402 on an end of the C-arm 101 and the opening 104b of the first air flow path 104 are included. The other end of the C-arm 101 has the opening 104a. The first air flow path 104 is formed by continuously connecting from the opening 104b to the opening 104a inside the C-arm 101.

As shown in FIGS. 6 and 7, inside the C-arm 101, the first rib portion 101b and the second rib portion 101c that divide a hollow space in the C-arm 101 are included. A hollow space of the C-arm 101 is divided into the first space 106a covered with the outer surface 101a and the first rib portion 101b, the second space 106b covered with the outer surface 101a, the first rib portion 101b, and the second rib portion 101c, and the third space 106c covered with the outer surface 101a and the second rib portion 101c. In the first embodiment, the first space 106a, the second space 106b, and the third space 106c are used as the first air flow path 104.

The cutout 105 is comprised of the facing surface 105a that enables to mount the X-ray tube device 102 by cutting out the outer surface 101a, the first rib portion 101b, and the second rib portion 101c on the surfaces facing the X-ray detector 103 and the inclined surface 105b that inclines toward the central direction of the arc of the C-arm 101 from the facing surface 105a.

As shown in FIGS. 8 and 9, the respective openings of the first space 106a, the second space 106b, and the third space 106c are formed also on the X-ray detector 103 side end of the C-arm 101. The first space 106a, the second space 106b, and the third space 106c are used as the first air flow path 104, and the X-ray detector 103 side end forms the opening 104a.

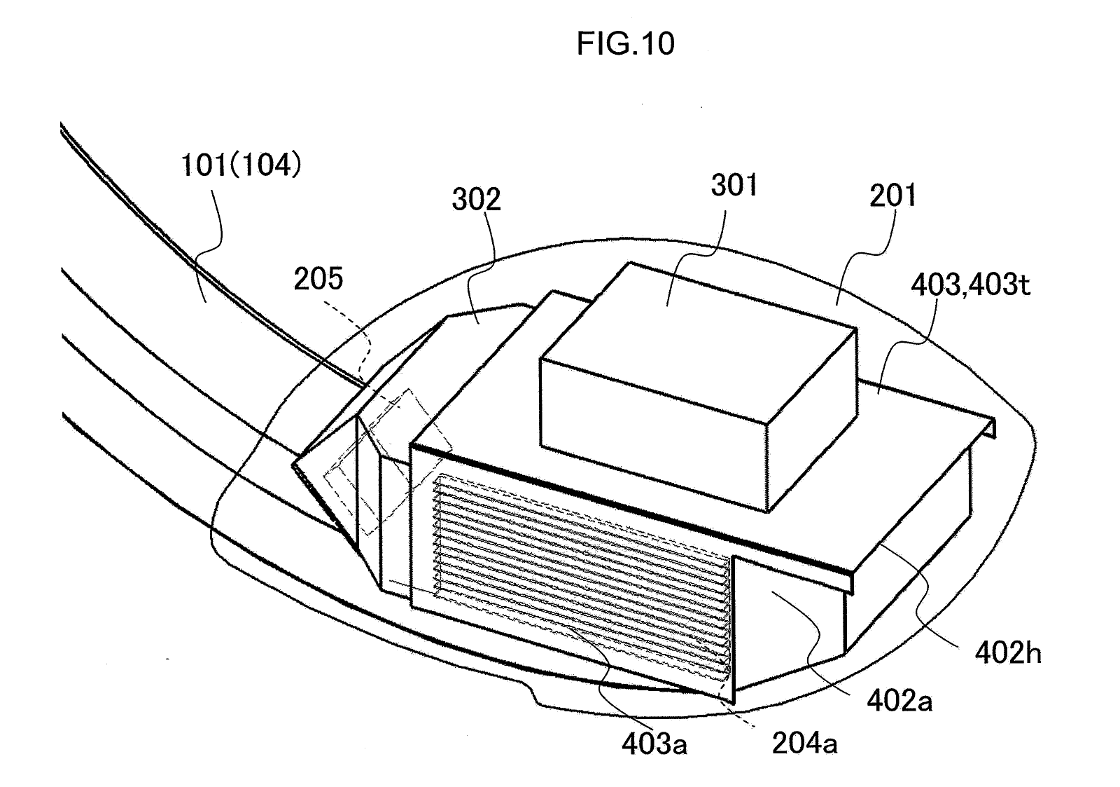

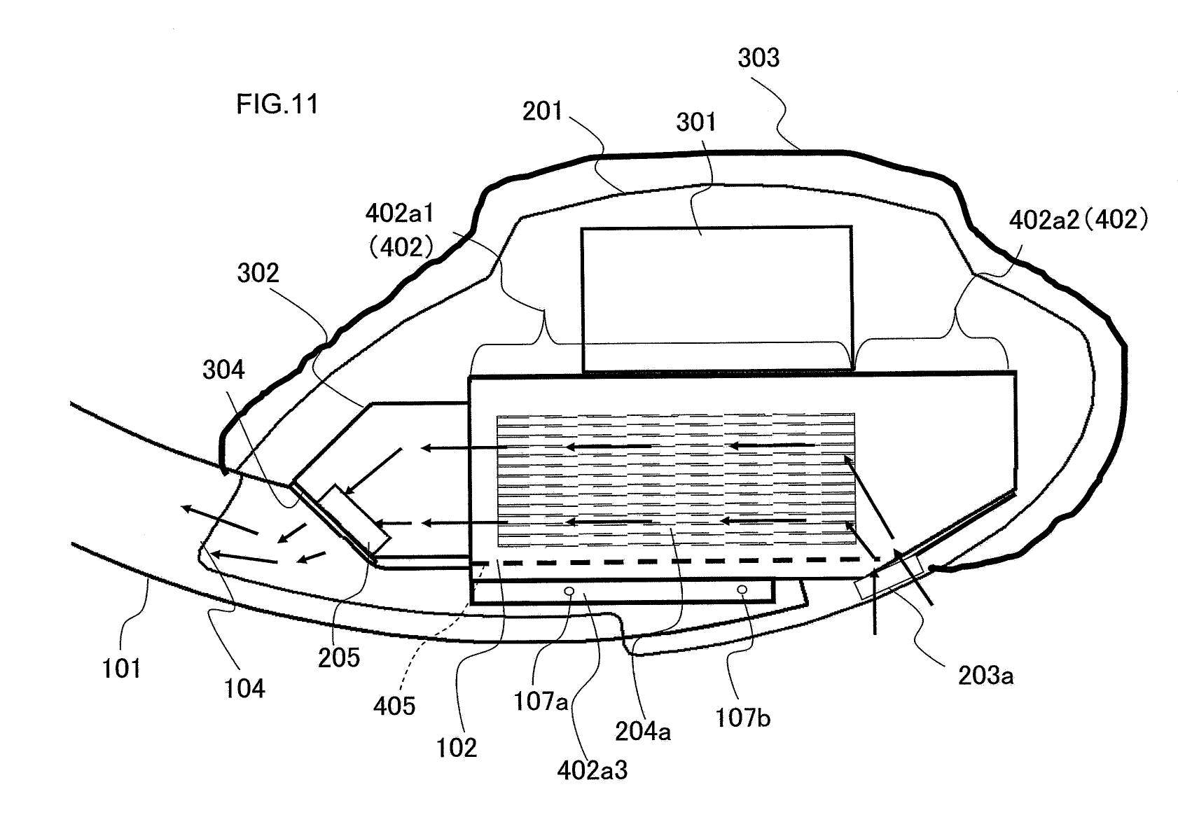

Next, based on FIGS. 10 to 14, the structure in the vicinity of an X-ray tube device in the mobile X-ray device 2 will be described. FIG. 10 is a perspective diagram showing an overall configuration of the vicinity of an X-ray tube device in the mobile X-ray device 2. FIG. 11 is a side-view diagram showing an overall configuration of an X-ray tube device housing and an air guide cover in the mobile X-ray device 2. FIG. 12 is a top-view diagram showing an overall configuration in the vicinity of an X-ray tube device in the mobile X-ray device 2.

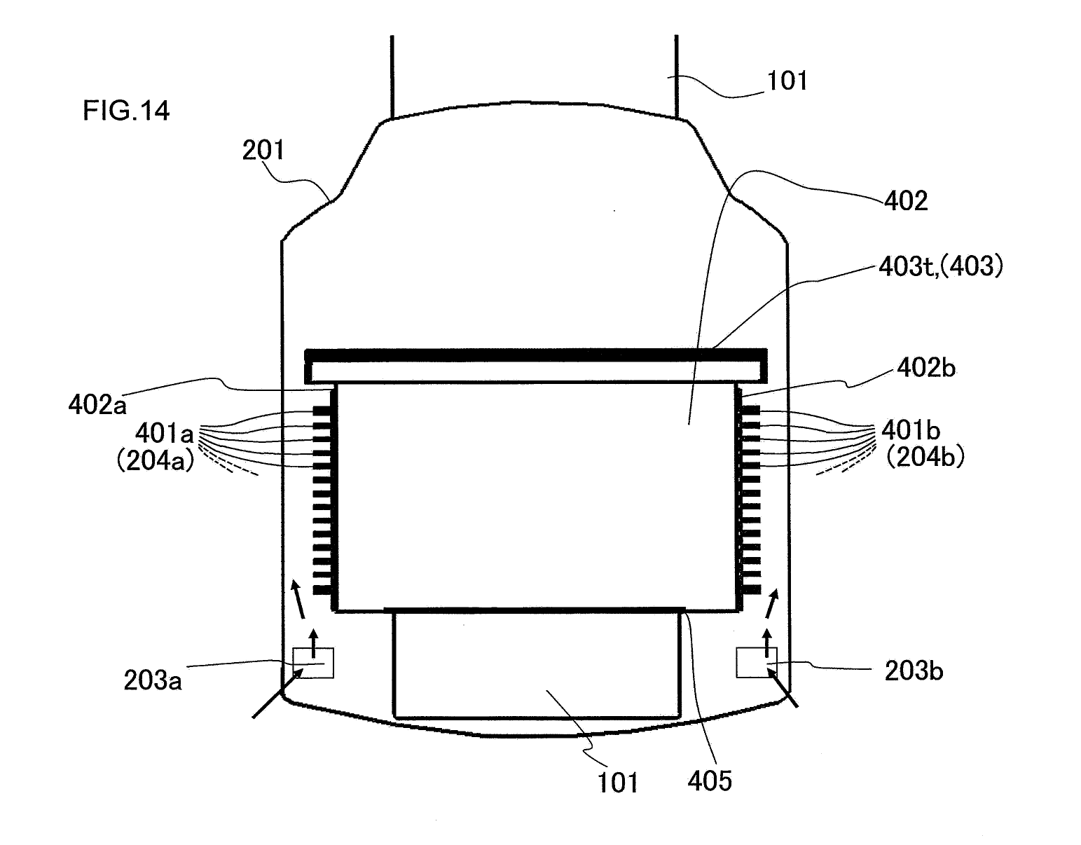

FIG. 13 is a front-view diagram showing an overall configuration in the vicinity of an X-ray tube device in the mobile X-ray device 2. FIG. 14 is an explanatory diagram showing an air flow path in a case where there are no bent portions of the housing cover. Additionally, the X-ray tube device cover 201 is opaque, and although the internal structure of the X-ray tube device cover 201 cannot be seen from the outside of the mobile X-ray device 2, for explanatory convenience, the inside of the X-ray tube device cover 201 is shown using solid lines in FIGS. 10 and 11.

As shown in FIG. 10, the housing cover 403 covers an opening surface of the X-ray tube device housing 402 and is comprised of a surface (hereinafter, referred to as “top surface”) 403t facing the X-ray detector 103, the first bent portion 403a that is bent from the top surface 403t and covers at least a part of the first side surface 402a of the X-ray tube device housing 402, and the second bent portion 403b that covers the second side surface 402b of the X-ray tube device housing 402. On the first side surface 402a of the X-ray tube device housing 402, the heat-release fin 204a to release heat generated from an X-ray tube is mounted. Actually, although the heat-release fin 204a cannot be seen from the outside of the housing cover 403, for explanatory convenience, the heat-release fin 204a is shown using dotted lines in FIG. 10. The X-ray diaphragm 301 is mounted on the X-ray detector 103 side on the top surface 403t of the housing cover 403 as well as between the X-ray radiation aperture 102c described before and the X-ray detector 103. The C-arm 101 side end of the X-ray tube device 102 and the first air flow path 104 are connected with the air guide cover 302.

As shown in FIG. 11, the first side surface 402a of the X-ray tube device housing 402 is comprised of the first region 402a1 that is approximately rectangular and the second region 402a2 having an approximately triangle cutout in the lower portion. The first region 402a1 stands from the bottom surface 402c of the X-ray tube device housing 402, and the second region 402a2 stands from the inclined surface 402e of the X-ray tube device housing 402. The heat-release fin 204a is molded integrally with the X-ray tube device housing 402 on the first side surface 402a of the X-ray tube device housing 402 that is made of a material with high heat conductivity such as aluminum. The overall dimension of the heat-release fin 204a is almost equivalent to the first region 402a1 of the first side surface 402a of the X-ray tube device housing 402. The above first bent portion 403a is configured so that the first region 402a1 of the first side surface 402a is covered.

The lower end of the first side surface 402a of the X-ray tube device housing 402 has the fixing part 402a3 protruded lower (toward the opposite direction to the X-ray detector 103) than the bottom surface 402c of the X-ray tube device housing 402. The fixing part 402a3 is fixed with the two fixing members 107a and 107b on the facing surface 105a of the cutout 105 of the C-arm 101. Similarly, the lower end of the second side surface 402b also has the fixing part protruded lower than the bottom surface 402c of the X-ray tube device housing 402 (not shown in the figure). Then, the facing surface 105a and the above fixing part are fixed with fixing members. At this time, the heat-release sheet 405 is placed on the facing surface 105a of the cutout 105 of the C-arm 101 and is fixed by mounting the X-ray tube device housing 402 on the sheet. It is more favorable for the heat-release sheet 405 to have an electrical insulation property, and for example, an acrylic heat-release sheet may be used.

As shown in FIG. 12, the second side surface 402b facing the first side surface 402a of the X-ray tube device housing 402 also has the heat-release fin 204b similarly to the first side surface 402a. Also, the housing cover 403 has the second bent portion 403b similarly to the first bent portion 403a.

As shown in FIG. 13, the heat-release fin 204a has the fin group 401a, and the heat-release fin 204b has the fin group 401b. The fin group 401a is configured so that an end contacts the first side surface 402a and the other end as an open end. The fin group 401b is configured so that an end contacts the first side surface 402b and the other end as an open end. The fin group 401a is comprised of a number of thin plates that stand toward the outside from the outer surface on the first side surface 402a of the X-ray tube device housing 402. The fin group 401b is comprised of a number of thin plates that stand toward the outside from the outer surface on the second side surface 402b of the X-ray tube device housing 402. These thin plates are mounted at almost regular intervals in the width directions (the w direction in FIG. 13) of air flow paths.

The top surface 403t of the housing cover 403 covers the opening surface 402g and the facing surface 402t of the X-ray tube device housing 402. Additionally, the housing cover 403 continues to the top surface 403t and has the first bent portion 403a facing the first side surface 402a of the X-ray tube device housing 402 at the interval d. The first bent portion 403a is formed from a rectangular plate member that has a size protruding (spreading out) in the first side surface 402a direction from the top surface 403t by perpendicularly bending the member from a long side of the top surface 403t.

Also, the housing cover 403 continues to the top surface 403t and has the second bent portion 403b facing the second side surface 402b of the X-ray tube device housing 402 at the interval d. The second bent portion 403b is formed from a rectangular plate member that has a size protruding (spreading out) in the second side surface 402b direction from the top surface 403t by perpendicularly bending the member from the other long side of the top surface 403t.

The first bent portion 403a contacts an open end of the fin group 401a on a surface (internal wall) facing the first side surface 402a. Similarly, the second bent portion 403b contacts an open end of the fin group 401b on a surface (internal wall) facing the second side surface 402b.

The second air flow paths 601a and 601b through which air passed through the fin groups 401a and 401b flows are formed between the first side surface 402a and the first bent portion 403a as well as between the second side surface 402b and the second bent portion 403b. The second air flow paths 601a and 601b and the first air flow path 104 are continuously connected via an internal space of the air guide cover 302. The second air flow paths 601a and 601b are continuously connected to an internal space of the X-ray tube device cover 201 on the opposite end to the first air flow path 104. The air guide cover 302 is mounted in a state where the cover covers from the first air flow path 104 side end in the second air flow paths 601a and 601b to the fan 205.

The respective open ends of the air guide cover 302 i.e., the lower side (the X-ray tube device housing 402 side) ends in the first air flow path 104 are inserted in the insides of the first bent portion 403a and the second bent portion 403b of the housing cover 403 (see FIG. 12). The connection structure of the air guide cover 302 and the C-arm 101 will be described later.

The fan 205 is installed between the first air flow path 104 and the air guide cover 302. That is, the fan 205 is located between the first air flow path 104 and the second air flow paths 601a and 601b. The fan 205 specifies a blowing direction in the first air flow path 104 in the C-arm 101 and is comprised of an electrical fan, for example. The fan 205 always operates while the main power supply of the mobile X-ray device 2 is turned ON. The control unit 23 controls operation and stoppage of the fan 205. Although the fan 205 cannot be seen from the outside of the air guide cover 302 because the fan 205 is inside the air guide cover 302, the fan 205 is shown in FIGS. 10, 11, and 12 for explanatory convenience.

Normally, when the mobile X-ray fluoroscopic imaging apparatus 1 is used in an operating room, the X-ray tube device cover 201 is covered with the sterilization cap 303. Therefore, even if the sterilization cap 303 is used, the cover openings 203a and 203b are disposed in positions where the sterilization cap 303 does not cover. Additionally, the cover opening 203a is disposed in the lower vicinity of the second air flow path 601a, and the cover opening 203b is disposed in the lower vicinity of the second air flow path 601b (see FIGS. 11 and 12).

The internal space of the X-ray tube device cover 201 is continuously connected to the external space of the X-ray tube device cover 201 via the cover openings 203a and 203b. Also, the cover openings 203a and 203b are blocked with a net plate (not shown in the figure), for example, so as not to touch the X-ray tube device 102 by accidentally putting fingers etc. in the cover openings 203a and 203b.

Function effects of the first bent portion 403a and the second bent portion 403b are described by comparing to a case without these portions. As shown in FIG. 14, if the first bent portion 403a and the second bent portion 403b are not provided, air flowed from the cover openings 203a and 203b passes around the heat-release fins 204a and 204b. Therefore, the state shown in FIG. 13 where the second air flow paths 601a and 601b are formed can enhance the air-cooling effect more than the state in FIG. 14.

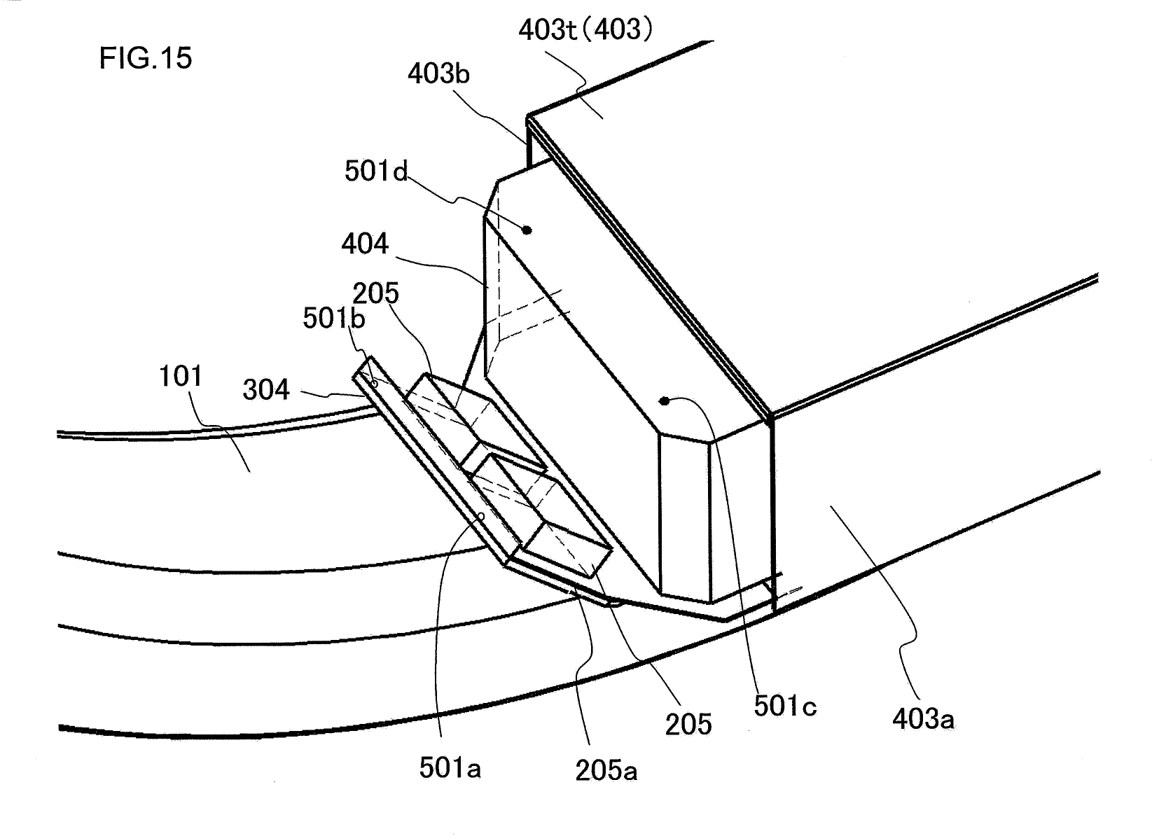

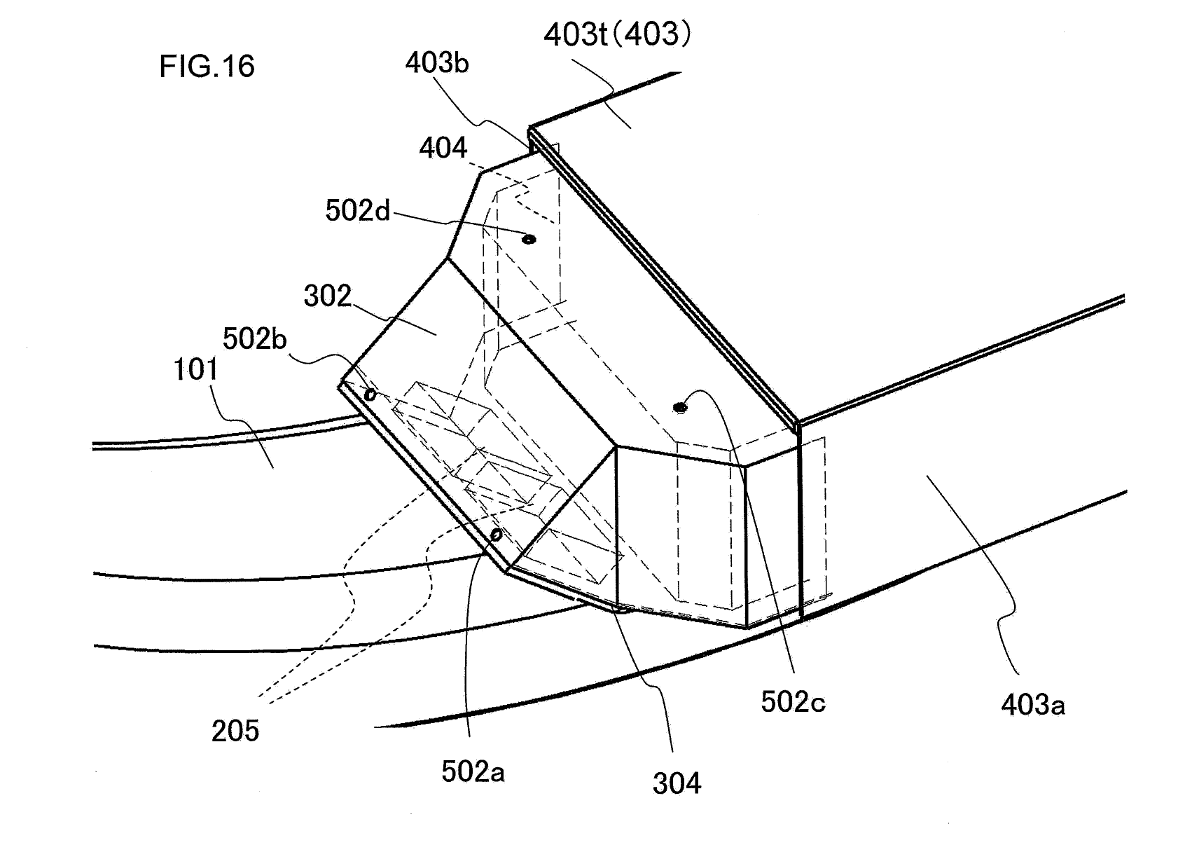

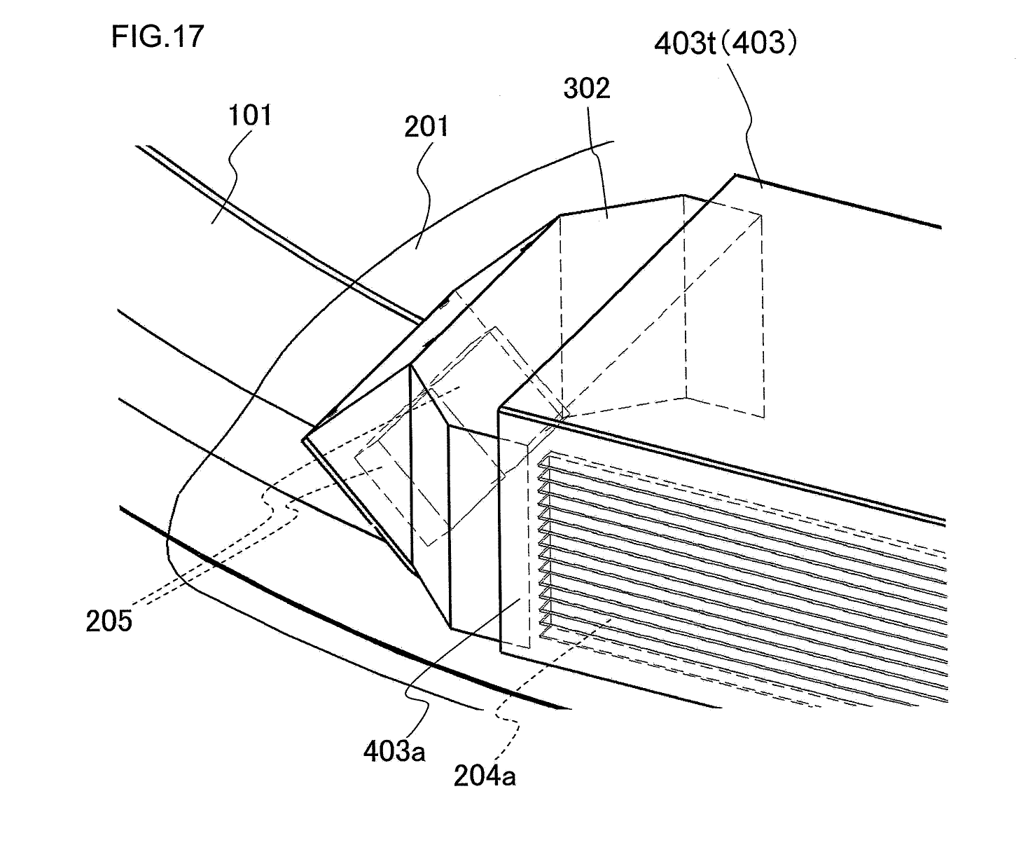

Based on FIGS. 15 to 17, the connection structure of the X-ray tube device 102 and the C-arm 101 will be described. FIG. 15 is a perspective diagram showing a structure in the vicinity of the X-ray tube device 102 of the C-arm 101 before an air guide cover is installed. FIG. 16 a perspective diagram showing a structure in the vicinity of the X-ray tube device 102 of the C-arm 101 after an air guide cover is installed and shows a state where the air guide cover 302 is viewed from the C-arm 101 side. FIG. 17 is a perspective diagram showing a structure in the vicinity of the X-ray tube device 102 of the C-arm 101 after an air guide cover is installed and shows a state where the air guide cover 302 is viewed from the X-ray tube device 102 side.

As shown in FIG. 15, on the open end 104b of the first air flow path 104 in the C-arm 101, the fan supporting portion 205a that supports the fan 205 is disposed. The fan supporting portion 205a is comprised of a metal plate, for example. On the X-ray tube device 102 side of the fan supporting portion 205a, the two fans 205 are fixed using fixing members such as screws. The fan supporting portion 205a has a blowing opening (not shown in the figure) to continuously connect the fan 205 and the first air flow path 104. Via the blowing opening, air flows between the open end 104b of the first air flow path 104 and the fan 205. On the C-arm 101 side surface of the fan supporting portion 205a, a member with insulating and sealing properties, such as the sponge 304 is adhered. Therefore, the fan supporting portion 205a and the inclined surface 105b of the cutout 105 of the C-arm 101 are connected across the sponge 304. Hence, a gap between the air guide cover 302 and the C-arm 101 is filled.

The two fixing holes 501a and 501b are provided on the X-ray detector 103 side of the fan supporting portion 205a.

The two fixing holes 501c and 501d are provided on the surface facing the X-ray detector 103 of the protection cover 404.

As shown in FIGS. 16 and 17, the air guide cover 302 is inserted so that the cover adhered inside the first bent portion 403a and the second bent portion 403b. Hence, a space from the lower side end surface (the cover openings 203a and 203b side end surface) of the second air flow paths 601a and 601b to the fan 205 is covered and sealed. Then, in the positions of the fixing holes 501a, 501b, 501c, and 501d, the air guide cover 302 and the fan supporting portion 205a are fixed using the fixing members 502a and 502b such as screws. Similarly, the air guide cover 302 and the protection cover 404 are fixed using the fixing members 502c and 502d such as screws.

The functions and effects of the mobile X-ray fluoroscopic imaging apparatus 1 related to the first embodiment will be described. Generally, although a mobile X-ray fluoroscopic imaging apparatus is used for fluoroscoping and imaging during a surgical operation, the apparatus is used in huge output and for a long time in many cases. In a case of using a mobile X-ray fluoroscopic imaging in huge output and for a long time apparatus, heat generated by an X-ray tube inside the X-ray tube device 102 conducts to the X-ray tube device housing 402 through internal insulating oil, resulting in an increase in temperature of the entire X-ray tube device 102.

According to the present embodiment, heat conducted to the X-ray tube device housing 402 conducts also to the first side surface 402a and the second side surface 402b of the X-ray tube device housing 402 as well as the heat-release fins 204a and 204b. Then, the fan 205 that always operates while the mobile X-ray fluoroscopic imaging apparatus 1 is operating allows outside air flowed from the cover openings 203a and 203b to always flow during the operation between the fin groups 401a and 401b of the heat-release fins 204a and 204b. Heat conducted to the heat-release fins 204a and 204b is thermally exchanged with air flowing between the fin groups 401a and 401b, enters in the air guide cover 302 from the lower air flow path, and then flows into the fan 205. Then, the heat is sent from the opening 104b to the first air flow path 104 inside the C-arm 101 by the fan 205. The air thermally exchanged and heated in the heat-release fins 204a and 204b reaches the X-ray detector 103 side end via the first air flow path 104 inside the C-arm 101. The air flowed to the X-ray detector 103 side end enters into the X-ray detector cover 202 from the opening 104a. Then, from the gap 108 between the X-ray detector cover 202 and the C-arm 101, the air is released little by little by leaking outside the X-ray detector cover 202.

In the process where air flows inside the C-arm 101, some heat in the heated air is thermally exchanged by a material with high thermal conductivity such as the internal surface of the C-arm 101 formed by aluminum. Heat-release efficiency can be improved by thermally exchanging with the C-arm 101 whose heat amount is significantly large. Then, the released exhaust air is cooled while passing through the inside of the C-arm 101, therefore, high-temperature exhaust air is prevented from being exhausted.

Also, according to the present embodiment, because the air flowed to the X-ray detector 103 side end is released little by little by leaking outside the device from the gap 108 of the X-ray detector cover 202, the air whose momentum is increased by the fan 205 does not directly flow to the outside of the device, resulting in preventing dust from raising. Also, the momentum of the exhausted air to be released can be reduced.

Also, open ends of the heat-release fins 204a and 204b cohere to the first bent portion 403a and the second bent portion 403b, and the air guide cover 302 covers a space from the lower side end of the air flow paths of the second air flow paths 601a and 601b to the fan 205. Additionally, the sponge 304 is used to fill the gap between the fan 205 and the C-arm 101. Therefore, the second air flow paths 601a and 601b and the first air flow path 104 are sealed. Accordingly, air that passed through the heat-release fins 204a and 204b to be thermally exchanged passes through the fan 205 and is sent only to the inside of the C-arm 101.

Also, although air passes not between the fin groups 401a and 401b having high pressure losses but between the heat-release fins 204a and 204b and the inner wall surface of the X-ray tube device cover 201 that are outside than open ends of the heat-release fins 204a and 204b and flows into the air guide cover 302 in a case without the first bent portion 403a and the second bent portion 403b, the present embodiment provides the first bent portion 403a and the second bent portion 403b to allow air to pass between the fin groups 401a and 401b much easier, resulting in improvement of heat-release efficiency.

Also, because an X-ray is irradiated to the housing cover 403 side, the insulating oil that is heated around an X-ray tube tends to flow to the housing cover 403 side in large amounts, and the housing cover 403 tends to be most-heated in the X-ray tube device housing 402. According to the present embodiment, because air flows also on the surface of the first bent portion 403a and the second bent portion 403b simultaneously with the fin groups 401a and 401b when passing through the heat-release fins 204a and 204b, thermal exchange can be directly performed with a part of the housing cover 403 that tends to be most-heated.

Also, because heat conducted to the housing cover 403 conducts to the air guide cover 302 that contacts the first bent portion 403a and the second bent portion 403b, air is to be thermally exchanged even on the surface of the air guide cover 302 when flowing inside the air guide cover 302.

Also, by connecting the air guide cover 302 and the C-arm 101 with a member having insulating and sealing properties such as the sponge 304 inserted between them, the X-ray tube device 102 and the C-arm 101 can be insulated, and reliability in safety is improved. Additionally, because installing the X-ray tube device 102 on the C-arm 101 is naturally and finely adjusted according to the crushing degree of the sponge 304, the air flow path can be easily sealed without increasing man-hours, resulting in improvement of heat-release efficiency.

Additionally, by inserting the heat-release sheet 405 between the X-ray tube device housing 402 and the C-arm 101, heat conducted to the X-ray tube device housing 402 can be directly conducted to the C-arm 101 having a larger heat amount, which can improve heat-release efficiency. In addition, in a case where an electric insulating property is provided for the heat-release sheet 405, reliability in safety can be improved by insulating the X-ray tube device housing 402 and the C-arm 101. If insulating without using the heat-release sheet 405 having an electric insulating property, the X-ray tube device 102 must be installed with it floating from the C-arm 101. However, by using a heat-release sheet having an electric insulating property, it is easier to install the device compared to the former case, which can reduce man-hours for the installation.

### Second Embodiment

Although the heat-release fins 204a and 204b are molded integrally with the X-ray tube device housing 402 in the first embodiment, the heat-release fins 204a and 204b are configured separately from the X-ray tube device housing 402 and fixed by contacting them on the outer surface of the X-ray tube device housing 402 in the second embodiment.

In the second embodiment, instead of integrally molding the heat-release fins 204a and 204b with the X-ray tube device housing 402, the first side surface 402a and the second side surface 402b of the X-ray tube device housing 402 are comprised of flat surfaces. Then, each one side of the heat-release fins 204a and 204b is comprised of a flat surface. The flat surfaces of the heat-release fins 204a and 204b are adhered to the first side surface 402a and the second side surface 402b.

According to the present embodiment, because the heat-release fins 204a and 204b do not need to be integrally molded on the side surfaces of the X-ray tube device housing 402, the X-ray tube device housing 402 can be manufactured easily compared to the first embodiment.

### Third Embodiment

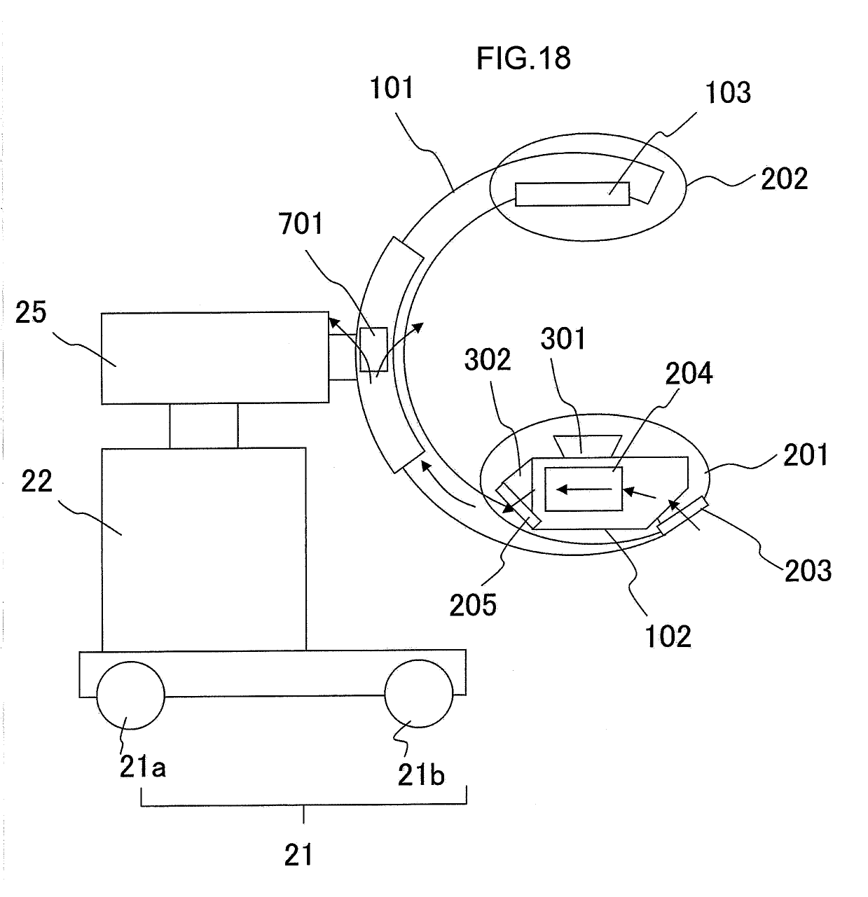

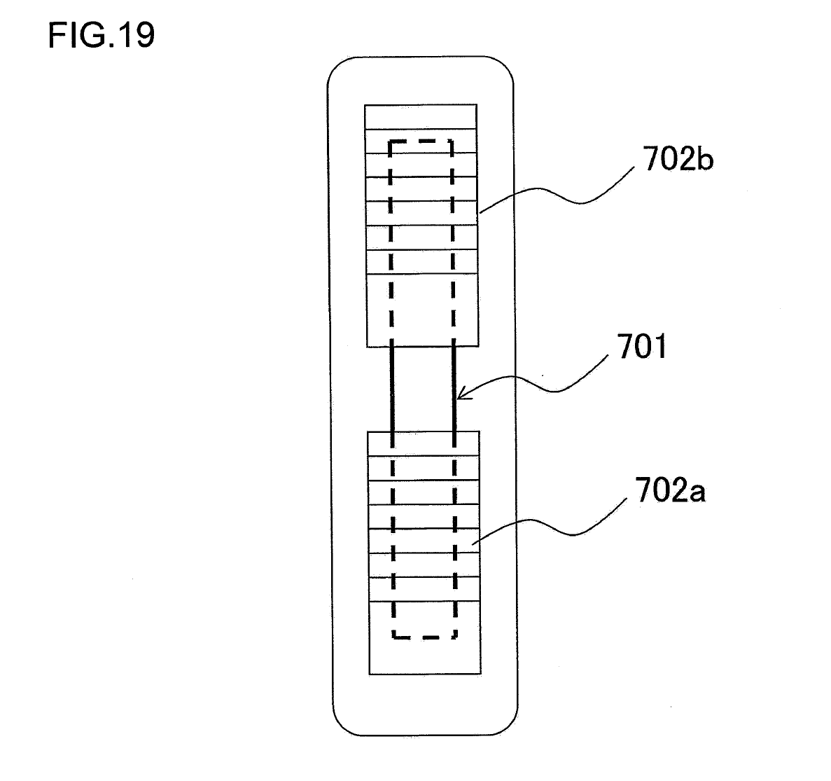

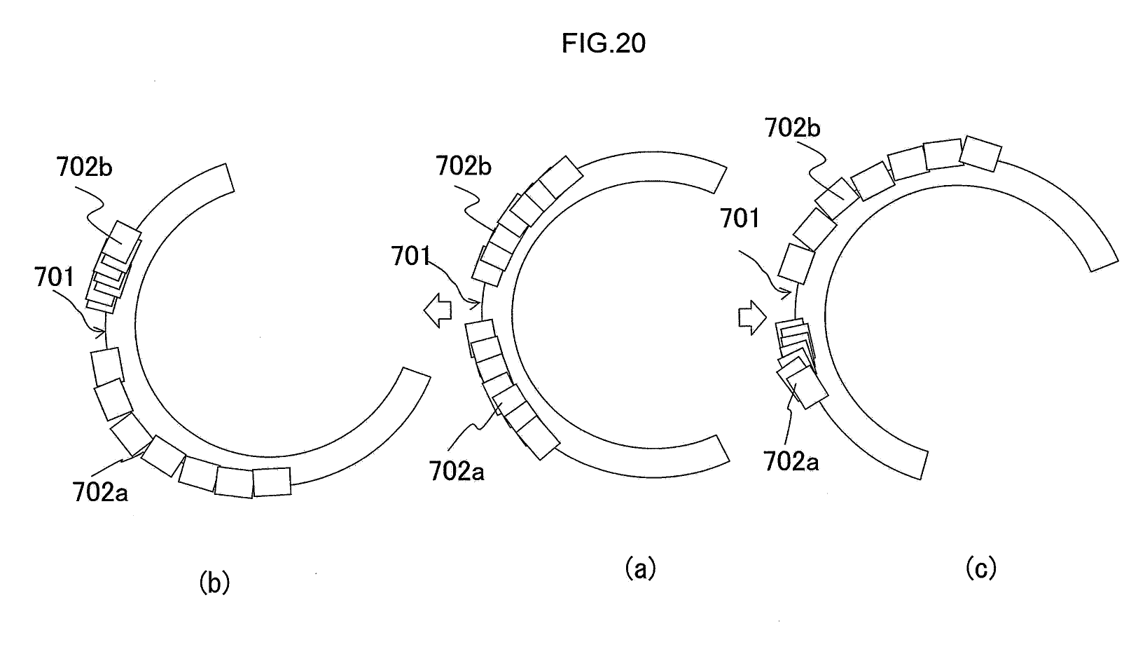

Although air sent to the inside of the C-arm 101 flows to the X-ray detector 103 side end of the C-arm 101 in the first embodiment, the third embodiment has a configuration where an exhaust outlet is provided in the middle of the C-arm 101 to emit the air from the outlet. Hereinafter, the third embodiment will be described based on FIGS. 18 to 20. FIG. 18 is an explanatory diagram showing an overview of a heat-release mechanism of a mobile X-ray fluoroscopic imaging apparatus related to the second embodiment. FIG. 19 is an explanatory diagram where the vicinity of an exhaust outlet in the C-arm 101 is magnified. FIG. 20 is an explanatory diagram showing a transformed state after the C-arm 101 is slid.

As shown in FIG. 18, in the third embodiment, the arm opening (hereinafter, referred to as “exhaust outlet”) 701 that continuously connects the first air flow path 104 inside the C-arm 101 to an external space of the C-arm 101 is provided in the vicinity of the arm supporting unit 25 for the C-arm 101. The first air flow path 104 is sealed to the exhaust outlet 701 and is not provided on the X-ray detector 103 side from the exhaust outlet 701. Then, air flowed from the cover openings 203a and 203b of the X-ray tube device cover 201 passes through the heat-release fins 204a and 204b, the air guide cover 302, and the first air flow path 104 and is emitted from the exhaust outlet 701 to the outside of the mobile X-ray device 2 (the arrows in FIG. 18 show the direction of the air flow).

As shown in FIG. 19, the outer surface 101 a on the X-ray detector 103 side across the exhaust outlet 701 of the C-arm 101 is comprised of the bellows section 702a. The outer surface 101 a on the X-ray tube device 102 side across the exhaust outlet 701 of the C-arm 101 is comprised of the bellows section 702b.

When the C-arm 101 slides in the arrow H direction shown in FIG. 1, according to this, the bellows sections 702a and 702b are stretched, and the exhaust outlet 701 is configured so that it is located at the almost same position before and after the slide movement of the C-arm 101.

FIG. 20 shows the slide movement of the C-arm 101 and the movement of the bellows sections 702a and 702b that are stretched according to the movement. In the state A in FIG. 20, both the bellows sections 702a and 702b are not stretched, the exhaust outlet 701 is located in the vicinity of the rotational center of the C-arm 101.

When sliding the C-arm 101 to the X-ray tube device 102 side along the arrow H direction in FIG. 1 from the state A, the bellows section 702b is collapsed, the bellows section 702a is expanded, resulting in the state B. Even in the state B, the position of the exhaust outlet 701 is not changed from that in the state A by transforming the bellows sections 702a and 702b.

Also, when sliding the C-arm 101 to the X-ray detector 103 side along the arrow H direction in FIG. 1 from the state A, the bellows section 702a is collapsed, the bellows section 702b is expanded, resulting in the state C. Even in the state C, the position of the exhaust outlet 701 is not changed from that in the states A and B by transforming the bellows sections 702a and 702b.

According to the present embodiment, because exhaust can be performed in a position that does not absolutely face a floor surface wherever the C-arm 101 is facing, dust and dirt are prevented from being raised due to the exhaust, an effect where use of a mobile X-ray fluoroscopic imaging apparatus in an operation room becomes more hygienic can be obtained.

### Forth Embodiment

Although a blowing direction of the fan 205 is always fixed in the first embodiment, the blowing direction of the fan 205 is controlled by a posture of the C-arm 101 in the forth embodiment, and the forth embodiment has a configuration where air always flows in the direction from a floor side to a ceiling side.

In a state where the C-arm 101 locates an X-ray tube device on a floor side and the X-ray detector 103 on a ceiling side, even if air leaks from the gap 108 of the X-ray detector cover 202, because the vertical distance from the floor is relatively larger than the X-ray tube device 102, concern where dust and dirt on the floor surface are raised due to the exhaust is reduced. However, the X-ray tube device 102 and the X-ray detector 103 may be located on the ceiling side and the floor side respectively by the C-arm 101 rotating in a vertical surface (rotating in the arrow C direction in FIG. 1). In this case, if air leaks from the gap 108 of the X-ray detector cover 202, concern where dust and dirt on the floor surface are raised due to the exhaust more easily compared to the above case is caused.

Therefore, providing a posture detection unit that detect a posture of the C-arm 101, based on the detection result, the control unit 23 controls a blowing direction in the first air flow path 104 so that the intake and exhaust are performed from an end closer to a floor side and an end closer to a ceiling side respectively of the X-ray tube device 102 side end and the X-ray detector 103 side end of the C-arm 101. The posture detection unit may detect a posture of the C-arm 101 based on a rotation amount and movement amount of the arm supporting unit 25, for example.

As the relevant configuration, for example, a rotation direction of the fan 205 is configured variably and may be changed according to the posture detected by the control unit 23. That is, the fan 205 a forward-backward rotatable fan, and the control unit 23 controls the fan 205 so that it rotates forward or backward based on the detection result.

For more details, if the X-ray tube device 102 and the X-ray detector 103 are located on a floor side and a ceiling side respectively, as described before, the control unit 23 makes the fan 205 rotate in a direction where air flows from the X-ray tube device 102 side end to the X-ray detector 103 side end in the first air flow path 104 of the C-arm 101 (hereinafter, referred to as “forward rotation”).

On the other hand, if the X-ray tube device 102 and the X-ray detector 103 may be located on a ceiling side and a floor side respectively, the control unit 23 makes the fan 205 rotate in a direction where air flows from the X-ray detector 103 side end to the X-ray tube device 102 side end in the first air flow path 104 of the C-arm 101 (hereinafter, referred to as “backward rotation”). In this case, air flows from the outside to the inside of the X-ray detector cover 202 through the gap 108 of the X-ray detector cover 202 located on the C-arm 101 and floor surface sides. Then, air flows from the opening 104a into the first air flow path 104 of the C-arm 101, passes through the fan 205 from the opening 104b, and then reaches the inside of the air guide cover 302.

Next, the air passes through the heat-release fins 204a and 204b from the air guide cover 302 and is exhausted from the cover openings 203a and 203b. Hence, regardless of a posture of the C-arm 101, air is exhausted from an end relatively further than a floor, and dust and dirt on a floor can be prevented from being raised due to the exhaust.

As an example of the other configuration to change a blowing direction, in case of using a fan that cannot rotate backward, the same number of fans that are two or more are installed oppositely, and the control unit 23 may detect a posture of the C-arm 101 and control so that only either of the fans are always operated in order to blow air from a floor surface side to a ceiling surface side. That is, the fan 205 is comprised of the first fan that blows air from the X-ray tube device 102 side end to the X-ray detector 103 side end and the second fan that blows air from the X-ray detector 103 side end to the X-ray tube device 102 side end in the first air flow path 104, and the control unit 23 may rotate the first fan or the second fan by selecting either one of them based on the result detected by the posture detection unit.

Additionally, providing a fan rotation supporting section that supports the fan 205 rotatably for a fixed element such as the inner surface of the first air flow path 104, the control unit 23 may change a blowing direction of the fan 205 by rotating the fan rotation supporting section based on a detection result of the C-arm 101 posture.

As described above, according to the present embodiment, regardless of the C-arm 101 posture, air can be exhausted from an end relatively further than a floor in the C-arm 101, and dust and dirt can be more prevented from being raised due to the exhaust.

### Fifth Embodiment

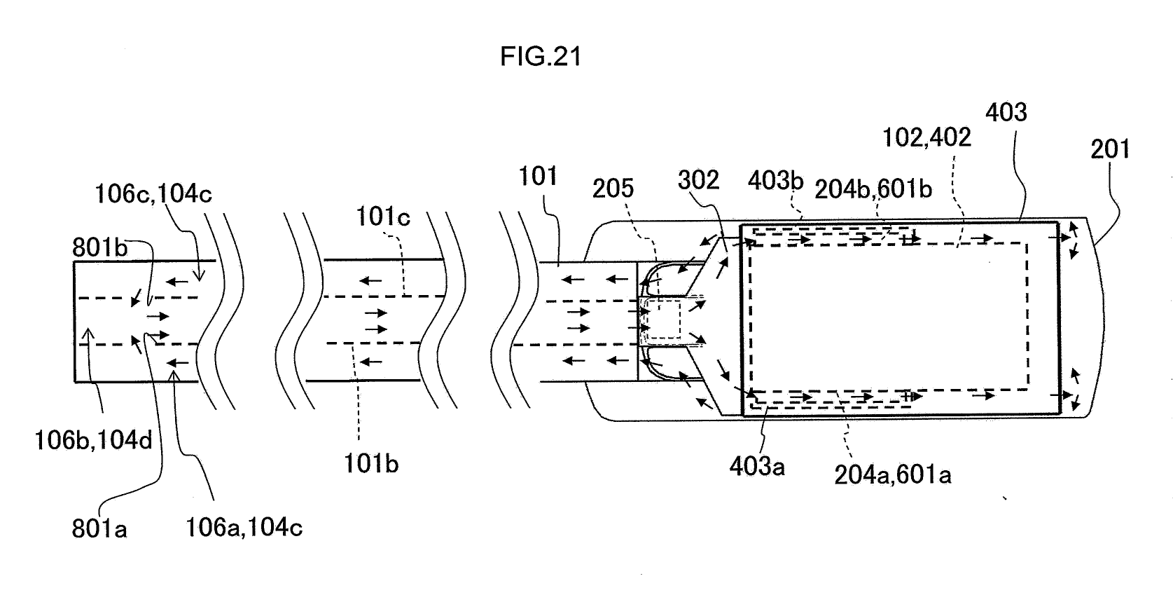

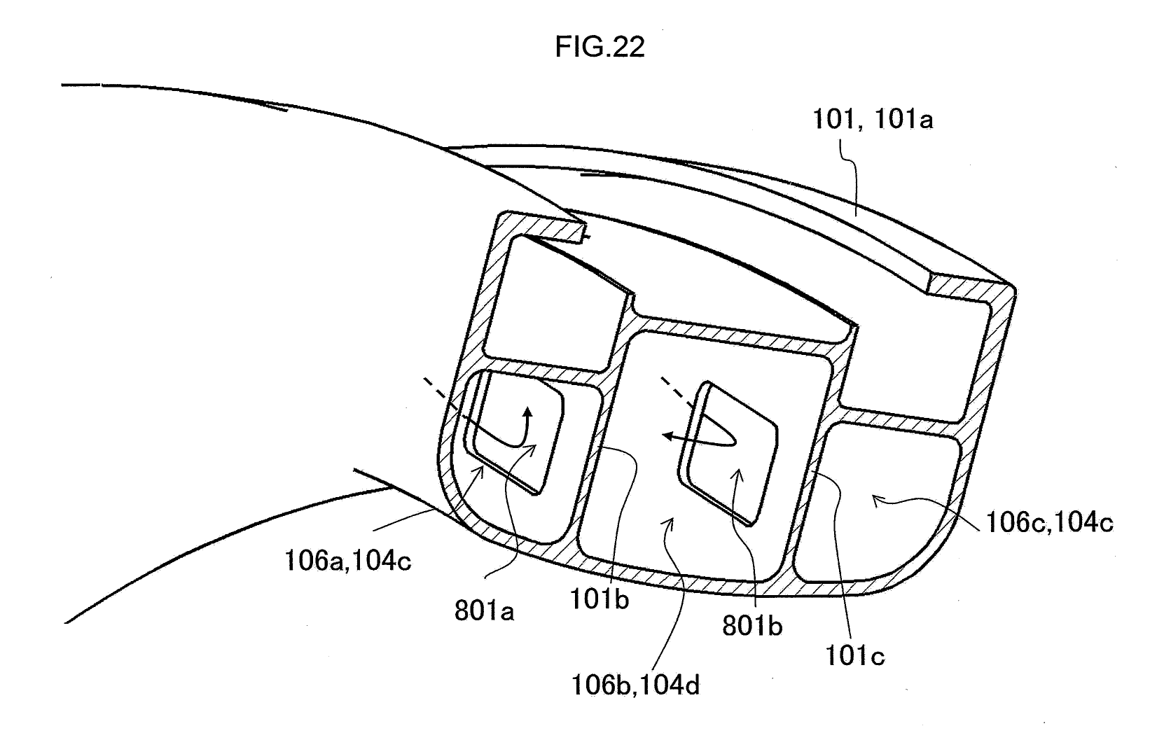

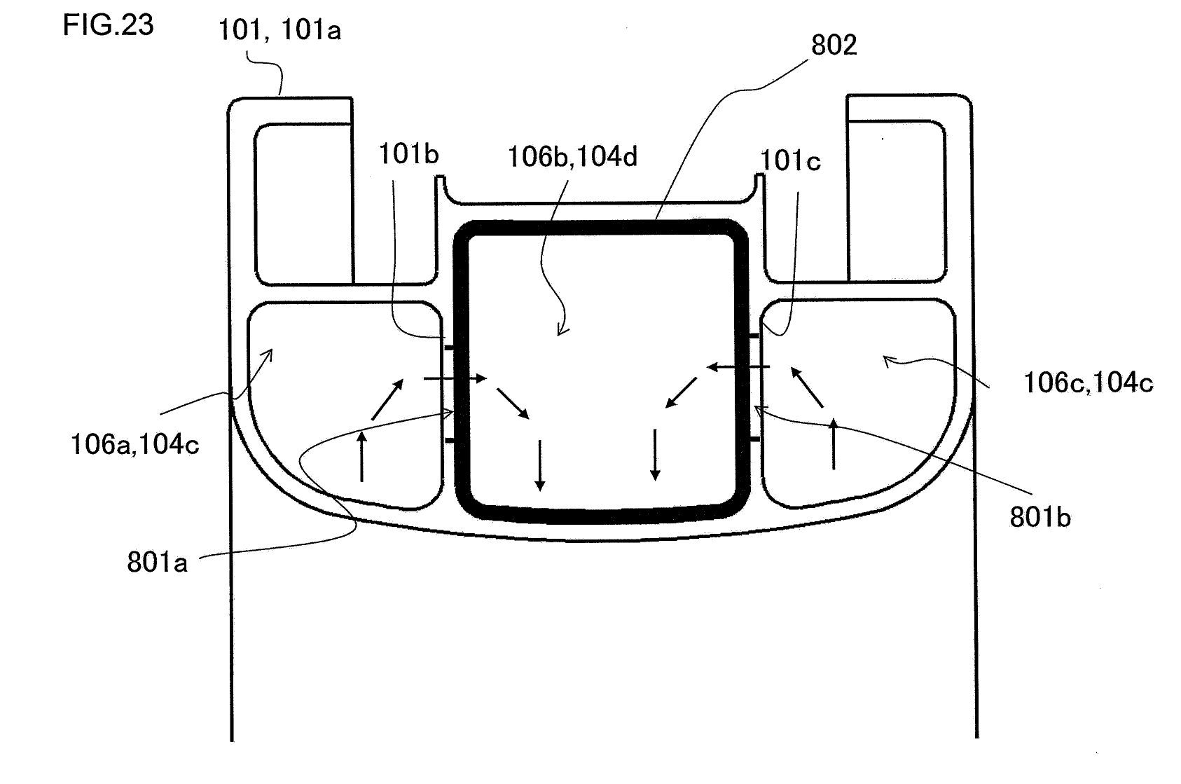

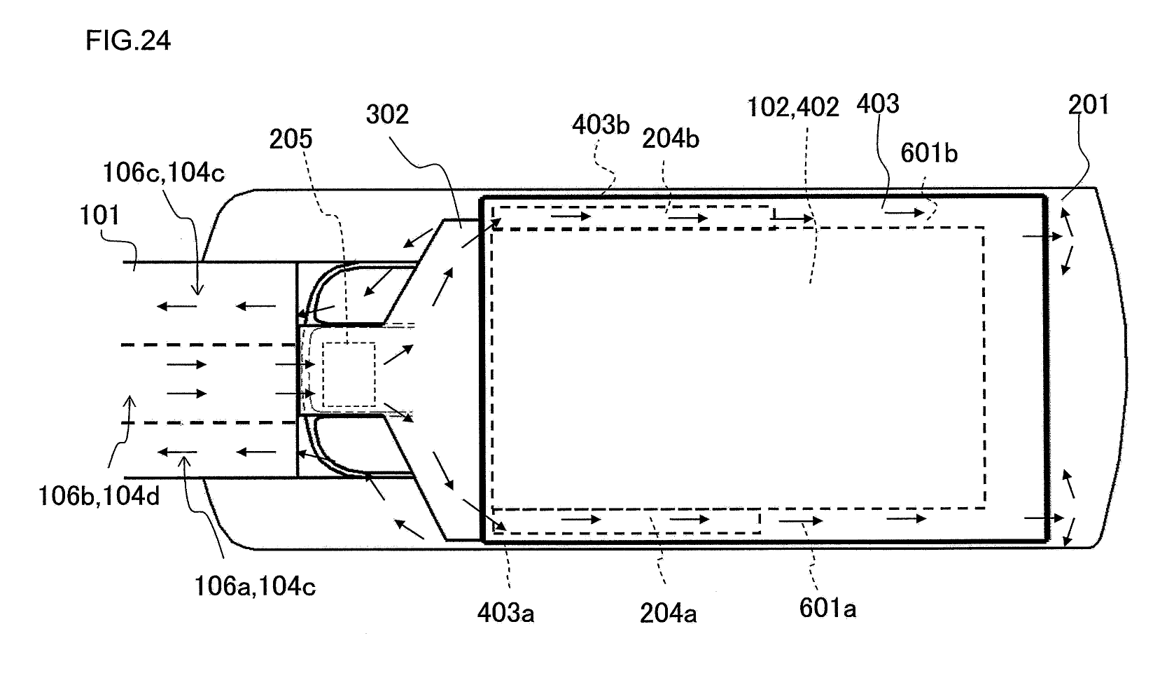

Although air is breathed from the outside of the mobile X-ray device 2, cools the heat-release fins 204a and 204b, and then is exhausted outside the apparatus in the first embodiment, air cools the heat-release fins 204a and 204b while circulating inside the mobile X-ray device 2 in the fifth embodiment. Hereinafter, the fifth embodiment will be described based on FIGS. 21 to 24. FIG. 21 is an explanatory diagram showing an overview of a heat-release mechanism of a mobile X-ray fluoroscopic imaging apparatus related to the fifth embodiment. FIG. 22 is a perspective diagram where the vicinity of an X-ray detector side in the C-arm 101 is magnified. FIG. 23 is a front-view diagram where the vicinity of an X-ray detector side in the C-arm 101 is magnified. FIG. 24 is an explanatory diagram where the vicinity of an X-ray tube device side in the C-arm 101 is magnified.

As shown in FIG. 21, the mobile X-ray device 2 related to the fifth embodiment has the air flow-in path 104c where air flows in the C-arm 101 from the X-ray tube device 102 and the air flow-out path 104d where air flows out from the inside of the C-arm 101 to the X-ray tube device 102 in the C-arm 101. In the fifth embodiment, the first space portion 106a and the third space portion 106c are used as the air flow-in path 104c, and the second space portion 106b is used as the air flow-out path 104d. The above air flow direction is an example. Therefore, the first space portion 106a and the third space portion 106c may be used as the air flow-out path 104d, and the second space portion 106b may be used as the air flow-in path 104c. In this case, the fan 205 rotates in a rotation direction that achieves a blowing direction where the first space portion 106a and the third space portion 106c are used as the air flow-out path 104d and the second space portion 106b is used as the air flow-in path 104c.

Also, in the vicinity of the X-ray detector cover 202 side end of the first rib portion 101b, the continuous connection hole 801a that continuously connects the first space portion 106a to the second space portion 106b is provided. Also, in the vicinity of the X-ray detector cover 202 side end of the second rib portion 101c, the continuous connection hole 801b that continuously connects the second space portion 106b to the third space portion 106c is provided.

On the X-ray tube device 102 side end of the C-arm 101, the air guide cover 302 is connected so that it is continuously connected only to the second space portion 106b. The first space portion 106a and the third space portion 106c are configured so that they are connected to the inside of the X-ray tube device cover 201.

The fan 205 is installed inside the air guide cover 302. The fan 205 rotates (rotates backward) in the direction where air is blown to the inside of the air guide cover 302 from the second space portion 106b. The installation position and rotation direction of the fan 205 are not limited to the above state if the fan 205 is installed inside the air flow-in path 104c or the air flow-out path 104d and if a blowing direction inside the second space portion 106b is toward the air guide cover 302 from the inside of the second space portion 106b. For example, if the fan 205 is located on the X-ray tube device 102 side end of the first space portion 106a and the third space portion 106c, the fan 205 may be rotated forward.

Additionally, because air circulates inside the mobile X-ray device 2 in the fifth embodiment, cover openings in the first embodiment and an exhaust outlet in the third embodiment are not provided.

As shown in FIG. 22, an insulation process is provided for the whole air flow-out path 104d on the inner wall surface of the second space portion 106b. The insulation process is performed by adhering the insulation sheet 802 made of polyester non-woven cloth etc. or providing an insulation coating on the inner wall surface, for example.

As shown in FIGS. 22 and 23, air passed through the first space portion 106a that is the air flow-in path 104c is blown into the second space portion 106b that is the air flow-out path 104d through the continuous connection hole 801a. Similarly, air passed through the third space portion 106c that is the air flow-in path 104c is blown into the second space portion 106b that is the air flow-out path 104d through the continuous connection hole 801b. When passing through the first space portion 106a and the third space portion 106c that are the air flow-in path 104c, air is cooled by the C-arm 101 housing. Although the cooled air is blown to the second space portion 106b, an insulation process is provided for the second space portion 106b and prevents heat from being conducted from the C-arm 101 housing to the cooled air.

As shown in FIG. 24, on the X-ray tube device 102 side end of the C-arm 101, air passes through the inside of the air guide cover 302 from the second space portion 106b that is the air flow-out path 104d and then passes through the heat-release fins 204a and 204b on the second air flow paths 601a and 601b. At this time, air absorbs heat generated from the X-ray tube device 102, resulting in air-cooling the heat-release fins 204a and 204b. From the lower end of the second air flow paths 601a and 601b to the inside of the X-ray tube device cover 201, air heated by the heat-release fins 204a and 204b is released. The heated air passes through the outside of the X-ray tube device housing 402 and the housing cover 403 and then flows into the first space portion 106a and the third space portion 106c of the C-arm 101. The flowed air is cooled by the C-arm 101 housing while passing through the first space portion 106a and the third space portion 106c and is blown into the second space portion 106b from the continuous connection holes 801a and 801b again.

According to a mobile X-ray fluoroscopic imaging apparatus related to the present embodiment, air heated by heat generated from the X-ray tube device 102 such as the heat-release fins 204a and 204b, the X-ray tube device housing 402, and the housing cover 403 is cooled by the C-arm 101 housing, and the cooled air is sent back to the X-ray tube device 102 while insulating in the C-arm 101, which can air-cool the X-ray tube device 102 without exhausting the air to the outside of the mobile X-ray device 2.

### Sixth Embodiment

Although the X-ray detector 103 is provided on the other end side of an arm in the above respective embodiments, the present invention can be applied also to an X-ray apparatus that has an X-ray detector on an end of an arm and that does not have an X-ray detector on the other end. That is, the present invention can be applied also to an X-ray apparatus using an X-ray detector and X-ray image receiving members that are configured separately from the mobile X-ray device 2 and not fixed to the arm, such as a portable FPD, an imaging plate, and an X-ray film for example.

Hence, air used to air-cool the X-ray tube device can be cooled when passing through the inside of the arm.

Although the fan 205 always operating while the main power source of the mobile X-ray device 2 is turned ON in the above embodiment, the fan 25 may be configured so that it can be stopped at an arbitrary timing. For example, a switch that can perform a switching operation to operate or stop manually may be provided. Also, the fan 205 may be configured to stop it when a posture of the C-arm is detected, and then the X-ray detector 103 exhausting air is detected to be near a floor. Hence, if a case where dust on the floor surface is raised due to the exhaust from the fan 205 is concerned, the fan 205 can be stopped manually or from a posture detection result.

### Reference Signs List

1: mobile X-ray fluoroscopic imaging apparatus, 2: mobile X-ray apparatus, 3: mobile image display device, 4: cable, 21: traveling unit, 21a and 21b: wheels, 22: main body, 23: control unit, 24: operation unit, 25: arm supporting unit, 31: image processing unit, 32: display, 101: C-arm (arm), 101a: outer surface, 101b: first rib portion, 101c: second rib portion, 102: X-ray tube device, 102a: X-ray tube, 102b: tube pipe, 102c: X-ray radiation aperture, 102d: outer member, 103: X-ray detector, 104: first air flow path, 104a and 104b: openings, 104c: air flow-in path, 104d: air flow-out path, 105: cutout, 105a: facing surface, 105b: inclined surface, 106a: first space portion, 106b: second space portion, 106c: third space portion, 107a and 107b: fixing members, 108: gap, 201: X-ray tube device cover, 202: X-ray detector cover, 203a and 203b: cover openings, 204a and 204b: heat-release fins, 205: fan, 205a: fan supporting portion, 301: X-ray diaphragm, 302: air guide cover, 303: sterilization cap, 304: sponge, 401a and 401b: fin groups, 402: X-ray tube device housing, 402a: first side surface, 402a1: first region, 402a2: second region, 402a3: fixing part, 402b: second side surface, 402c: bottom surface, 402e: inclined surface, 402g: opening surface, 402h: third side surface, 402i: forth side surface, 402t: facing surface, 403: housing cover, 403a: first bent portion, 403b: second bent portion, 403t: facing surface (top surface), 404: protection cover, 405: heat-release sheet, 501a, 501b, 501c, and 501d: fixing holes, 502a, 502b, 502c, and 502d: fixing members, 601a and 601b: second air flow paths, 701: exhaust outlet, 702a and 702b: bellows sections, 801a and 801b: continuous connection holes

1. An X-ray apparatus comprising:
an X-ray tube device having an X-ray tube and an X-ray tube device housing that accommodates the X-ray tube; an X-ray detector detecting an X-ray generated from the X-ray tube device; and an arm supporting the X-ray tube device on an end side and the X-ray detector on the other end side with them facing each other,
wherein a first air flow path where air that passed through the outer surface of the X-ray tube device housing flows is formed inside the arm.

2. The X-ray apparatus according to claim 1, comprising:
a heat-release fin having a fin group where an end contacts an outer surface and where the other end is an open end on the outer surface of the X-ray tube device housing,
wherein a second air flow path that is a flow path where air passes through the fin group and that is continuously connected to the first air flow path is formed in the X-ray tube device.

3. The X-ray apparatus according to claim 2, further comprising:
a housing cover covering a surface facing the X-ray detector in the X-ray tube device housing; and
an open end contact portion contacting an open end of the fin group on a surface facing the X-ray tube device of the housing cover,
wherein the second air flow path is formed between the surface of the X-ray tube device having the heat-release fin and the surface including the open end contact portion of the housing cover.

4. The X-ray apparatus according to claim 3, further comprising:
an air guide cover covering from the first air flow path side end of the second air flow path to the X-ray tube device side end of the first air flow path.

5. The X-ray apparatus according to claim 4, further comprising:
a fan being installed between the second air flow path and the first air flow path as well as specifying a blowing direction in the first air flow path;
a fan supporting portion supporting the fan,
the fan supporting portion is installed on the first air flow path side end of the air guide cover, and the fan supporting portion and the first air flow path end are connected with a member having insulating and sealing properties inserted.

6. The X-ray apparatus according to claim 3, comprising:
an X-ray tube device cover covering the X-ray tube device on an end of the arm; and
a cover opening continuously connecting an internal space of the X-ray tube device cover to an external space of the X-ray tube device cover,
wherein the second air flow path is continuously connected to an internal space of the X-ray tube device cover on an opposite end to the first air flow path side of the second air flow path, and
the first air flow path is continuously connected to an internal space of the second air flow path and the X-ray tube device cover as well as an external space of the X-ray tube device cover via the cover opening.

7. The X-ray apparatus according to claim 6, comprising:
a gap where air flows between the X-ray detector cover covering the X-ray detector and the arm being provided on the other end of the arm; and
an opening where the first air flow path is continuously connected to an internal space of the X-ray detector cover on the X-ray detector side end of the first air flow path,
wherein the first air flow path is continuously connected to an external space of the X-ray detector cover via the opening, an internal space of the X-ray detector cover, and the gap.

8. The X-ray apparatus according to claim 7, further comprising:
an arm supporting unit supporting the arm rotatably in order to reverse the vertical-direction positions of the X-ray tube device and the X-ray detector;
a fan specifying a blowing direction in the first air flow path;
a posture detection unit detecting a posture of the arm; and
a control unit controlling a blowing direction of the fan so that air is blown toward a side close to a ceiling from a position side relatively close to a floor in the first air flow path based on a detection result by the posture detection unit.

9. The X-ray apparatus according to claim 8,
wherein the fan is a forward-backward rotatable fan,
the control unit performs switching control for the forward rotation or backward rotation of the fan based on the detection result.

10. The X-ray apparatus according to claim 8,
wherein the fan includes a first fan blowing air in the first air flow path from the X-ray tube device side to the X-ray detector side and a second fan blowing air from the X-ray detector side to the X-ray tube device side, and
the control unit rotates the first fan or the second fan by selecting either one of them based on the detection result.

11. The X-ray apparatus according to claim 8, further comprising:
a fan rotation supporting section supporting the fan rotatably in order to reverse the blowing direction for a fixed element that fixes the fan,
wherein the control unit performs rotation control for the fan rotation supporting section based on the detection result.

12. The X-ray apparatus according to claim 6, further comprising:
an arm supporting unit, an arm opening, and a bellows section,
wherein the arm is an arc-shaped C-arm supporting the X-ray tube device and the X-ray detector with them facing each other,
the arm supporting unit supports the C-arm slidably along the arc,
the arm opening continuously connects the first air flow path to an external surface of the C-arm in the vicinity of the arm supporting unit of the C-arm, and
the bellows sections stretch with the slide movement to the X-ray tube device side and the X-ray detector side respectively with the arm opening centered.

13. The X-ray apparatus according to claim 3, further comprising:
an X-ray tube device cover covering the X-ray tube device, and
a fan specifying a blowing direction in the first air flow path,
wherein the inside of the arm has the first air flow path and the third air flow path that is separated from the first air flow path and that is continuously connected to an internal space of the X-ray tube device cover,
the first air flow path and the third air flow path are sealed so as not to continuously connect to the outside of the arm,
an insulation process is provided on either surface of the first air flow path and the third air flow path, and
the X-ray detector side end in the inside of the arm has a continuous connection portion that is connected to the first air flow path and the third air flow path.

14. The X-ray apparatus according to claim 1, comprising:
a main body having the arm; and
a traveling unit moving the main body on a floor surface.

15. The X-ray image diagnostic apparatus, comprising:
any one of X-ray apparatuses in claim 1;
an image processing unit generating an X-ray image based on an X-ray detected by the X-ray detector; and
an image display unit displaying the X-ray image.
